SEQUENCE LISTING (5 sequences)

SEQ ID NO: 1 - protein, 223 aa, synthetic construct [sequence omitted]

SEQ ID NO: 2 - protein, 432 aa, synthetic construct
  REGION 1..432: note = Synthetic
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 3 - protein, 6 aa, synthetic construct
  REGION 1..6: note = Synthetic
HHHHHH

SEQ ID NO: 4 - protein, 5 aa, synthetic construct
  REGION 1..5: note = Synthetic
DDDDK

SEQ ID NO: 5 - protein, 6 aa, synthetic construct
  REGION 1..6: note = Synthetic
ADDDDK

(19) United States
(12) Patent Application Publication
(10) Pub. No.: US 2024/0369579 A1
(43) Pub. Date: Nov. 7, 2024

(54) LATERAL FLOW METHODS, ASSAYS, AND DEVICES FOR DETECTING THE PRESENCE OR MEASURING THE AMOUNT OF UBIQUITIN CARBOXY-TERMINAL HYDROLASE L1 AND/OR GLIAL FIBRILLARY ACIDIC PROTEIN IN A SAMPLE

(71) Applicant: Abbott Laboratories, Abbott Park, IL (US)

(72) Inventors: Jamie Becnel, Abbott Park, IL (US); Saul A. Datwyler, Abbott Park, IL (US); Stacey Pazar Huth, Abbott Park, IL (US); Beth McQuiston, Abbott Park, IL (US); Jackie A. Garritty, Abbott Park, IL (US); Evan McCormick, Abbott Park, IL (US)

(21) Appl. No.: 18/766,065

(22) Filed: Jul. 8, 2024

Related U.S. Application Data
(63) Continuation of application No. PCT/US2023/061894, filed on Feb. 3, 2023.
(60) Provisional application No. 63/482,808, filed on Feb. 2, 2023.
(60) Provisional application No. 63/435,834, filed on Dec. 29, 2022.
(60) Provisional application No. 63/306,788, filed on Feb. 4, 2022.

(51) Int. Cl. G01N 33/68; G01N 33/543; G01N 33/573

(52) U.S. Cl. CPC G01N 33/6896; G01N 33/54388; G01N 33/573; G01N 2333/948; G01N 2800/28

(57) ABSTRACT

Disclosed herein are methods and devices for performing at least one lateral flow assay on a biological sample obtained from a subject to determine an amount or presence of ubiquitin carboxy-terminal hydrolase L1 (UCH-L1) alone, or an amount or presence of UCH-L1 and an amount or presence of glial fibrillary acidic protein (GFAP).

### RELATED APPLICATION INFORMATION

This application is a continuation of International Application No. PCT/US2023/061894, filed on Feb. 3, 2023, which claims priority to U.S. Application No. 63/306,788 filed on Feb. 4, 2022, U.S. Application No. 63/435,834, filed on Dec. 29, 2022, and U.S. Application No. 63/482,808, filed on Feb. 2, 2023, the contents of each of which are herein incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 7,938 Byte XML file named “14635USC1_ST26.XML,” created on Jul. 8, 2024.

### TECHNICAL FIELD

The present disclosure relates methods for performing at least one lateral flow assay on a biological sample obtained from a subject to determine an amount or presence of ubiquitin carboxy-terminal hydrolase L1 (UCH-L1), glial fibrillary acidic protein (GFAP), or an amount or presence of UCH-L1 and an amount or presence of GFAP.

### BACKGROUND

More than 5 million mild traumatic brain injuries (TBIs) occur each year in the United States alone. Currently, there is no simple, objective, accurate measurement available to help in patient assessment. In fact, much of TBI evaluation and diagnosis is based on subjective data. Unfortunately, objective measurements such as head CT and Glasgow Coma Scale (GCS) score are not very comprehensive or sensitive in evaluating mild TBI. Moreover, a head CT is unrevealing a vast majority of the time for mild TBI, is expensive, and exposes the patient to unnecessary radiation. Additionally, a negative head CT does not mean the patient has been cleared from having a concussion; rather it just means certain interventions, such as surgery are not warranted. Clinicians and patients need objective, reliable information to accurately evaluate this condition to promote appropriate triage and recovery. To date, limited data have been available for the use of UCH-L1 and GFAP in the acute care setting to aid in patient evaluation and management. To date, limited data have been available for the use of UCH-L1 and GFAP in the acute care setting to aid in patient evaluation and management.

Mild TBI or concussion is much harder to objectively detect and presents an everyday challenge in emergency care units globally. Concussion usually causes no gross pathology, such as hemorrhage, and no abnormalities on conventional computed tomography scans of the brain, but rather rapid-onset neuronal dysfunction that resolves in a spontaneous manner over a few days to a few weeks. Approximately 15% of mild TBI patients suffer persisting cognitive dysfunction. There is an unmet need for detecting and assessing mild TBI victims on scene, in emergency rooms and clinics, in the sports area and in military activity (e.g., combat).

Current algorithms for assessment of the severity of brain injury include Glasgow Coma Scale score and other measures. These measures may at times be adequate for relating acute severity but are insufficiently sensitive for subtle pathology which can result in persistent deficit. GCS and other measures also do not enable differentiation among types of injury and may not be adequate. Thus, patients grouped into a single GCS level entering a clinical trial may have vastly heterogeneous severity and type of injury. Because outcomes also vary accordingly, inappropriate classification undermines the integrity of a clinical trial. Improved classification of injury will enable more precise delineation of disease severity and type for TBI patients in clinical trials.

Additionally, current brain injury trials rely on outcome measures such as Glasgow Outcome Scale Extended, which capture global phenomena but fail to assess for subtle differences in outcome. Sensitive outcome measures are needed to determine how well patients have recovered from brain injury to test therapeutics and prophylactics.

### SUMMARY

In one embodiment, the present disclosure relates to a method comprising:
performing at least one lateral flow assay on a biological sample obtained from a subject to determine an amount or presence of ubiquitin carboxy-terminal hydrolase L1 (UCH-L1), glial fibrillary acidic protein (GFAP), or an amount or presence of UCH-L1 and an amount or presence of GFAP; and
displaying the amount or presence of UCH-L1, GFAP, or UCH-L1 and GFAP determined in the sample. In some embodiments of the above method, the at least one lateral flow assay is part of a lateral flow device. In still further embodiments of the above method, the lateral flow device comprises (a) at least one test strip; or (b) at least two test strips.

In some embodiments of the above method, (a) the lateral flow assay for UCH-L1 comprises a first specific binding partner which binds to an epitope on UCH-L1 and a second specific binding partner comprising a detectable label; and (b) the lateral flow assay for GFAP comprises a third specific binding partner which binds to an epitope on GFAP and a fourth specific binding partner comprising a detectable label. In yet further embodiments, the first specific binding partner, second specific binding partner, third specific binding partner, fourth specific binding partner, or any combination thereof is an antibody or antibody fragment thereof.

In yet other embodiments, the method is used to aid in a diagnosis and evaluation of a subject that has sustained or may have sustained an injury to the head. For example, in other embodiments, the subject is diagnosed as having a traumatic brain injury. When the subject is diagnosed as having a traumatic brain injury, the subject can be further diagnosed as having a mild, moderate, severe, or moderate to severe traumatic brain injury.

In still further embodiments of the above method, the method can be used to determine whether a subject should receive a head computed tomography (CT) scan, a magnetic resonance imaging (MRI) scan or both a head CT scan and a MRI scan.

In still yet further embodiments, the subject is a human subject (e.g., an adult subject and/or a pediatric subject).

In still yet further embodiments of the above method, the biological sample is the sample is selected from the group consisting of a whole blood sample, a urine sample, a cerebrospinal fluid sample, a tissue sample, a bodily fluid sample, a saliva sample, an oropharyngeal specimen, and a nasopharyngeal specimen. For example, in some embodiments, the sample is a whole blood sample. In other embodiments, the sample is a plasma sample. In still further embodiments, the sample is a serum sample. In yet still further embodiments, the sample is a saliva sample. In still yet further embodiments, the sample is urine sample. In still yet other embodiments, the sample is an oropharyngeal sample. In yet other embodiments, the sample is a whole blood sample and the subject is a human subject (e.g., an adult and/or pediatric subject). In other embodiments, the sample is a plasma sample and the subject is a human subject (e.g., an adult and/or pediatric subject). In still further embodiments, the sample is a serum sample and the subject is a human subject (e.g., an adult and/or pediatric subject). In yet still further embodiments, the sample is a saliva sample and the subject is a human subject (e.g., an adult and/or pediatric subject). In still yet further embodiments, the sample is urine sample and the subject is a human subject (e.g., an adult and/or pediatric subject). In still yet other embodiments, the sample is an oropharyngeal sample and the subject is a human subject (e.g., an adult and/or pediatric subject).

In yet further embodiments of the above method, the at least one lateral flow assay for GFAP, the at least one lateral flow assay for UCH-L1, or at least one lateral flow assay for GFAP and at least one lateral flow assay for UCH-L1 are each performed or capable of being performed in less than about 30 minutes, less than about 25 minutes, less than about 20 minutes, less than about 18 minutes, or less than about 15 minutes. In yet still further embodiments of the above method, the at least one lateral flow assay for GFAP, the at least one lateral flow assay for UCH-L1, or at least one lateral flow assay for GFAP and at least one lateral flow assay for UCH-L1 are each are each performed or capable of being performed in a time ranging from about 4 to about 20 minutes, from about 10 to about 15 minutes or from about 15 to about 18 minutes.

In another embodiment, the present disclosure relates to a kit for performing the above method. The kit can comprise: (a) a first lateral flow device for detecting a presence of ubiquitin carboxy-terminal hydrolase L1 (UCH-L1) in the sample; (b) a second lateral flow device for detecting a presence of glial fibrillary acidic protein (GFAP) in the sample; and (c) instructions for detecting the presence of UCH-L1 and GFAP in the sample.

In another embodiment, the present disclosure relates to a kit for performing the above method. The kit can comprise: (a) a lateral flow device for detecting a presence of ubiquitin carboxy-terminal hydrolase L1 (UCH-L1) and glial fibrillary acidic protein (GFAP) in the sample; and (b) instructions for detecting the presence of UCH-L1 and GFAP in the sample.

In another embodiment, the present disclosure relates to a method comprising:
performing at least one lateral flow assay on a biological sample obtained from a subject to determine an amount or presence of ubiquitin carboxy-terminal hydrolase L1 (UCH-L1), glial fibrillary acidic protein (GFAP) or both UCH-L1 and GFAP; and
visualizing the amount or presence of UCH-L1, GFAP, UCH-L1 and GFAP determined in the sample,
wherein the assay does not require a device to read the amount or presence of UCH-L1, GFAP, or UCH-L1 and GFAP (e.g., a reader or reading device).

In some embodiments of the above method, (a) the lateral flow assay for UCH-L1 comprises a first specific binding partner which binds to an epitope on UCH-L1 and a second specific binding partner comprising a detectable label; and (b) the lateral flow assay for GFAP comprises a third specific binding partner which binds to an epitope on GFAP and a fourth specific binding partner comprising a detectable label. In yet further embodiments, the first specific binding partner, second specific binding partner, third specific binding partner, fourth specific binding partner, or any combination thereof is an antibody or antibody fragment thereof.

In yet other embodiments, the method is used to aid in a diagnosis and evaluation of a subject that has sustained or may have sustained an injury to the head. For example, in other embodiments, the subject is diagnosed as having a traumatic brain injury. When the subject is diagnosed as having a traumatic brain injury, the subject can be further diagnosed as having a mild, moderate, severe, or moderate to severe traumatic brain injury.

In still further embodiments of the above method, the method can be used to determine whether a subject should receive a head computed tomography (CT) scan, a magnetic resonance imaging (MRI) scan or both a head CT scan and a MRI scan.

In still yet further embodiments, the subject is a human subject (e.g., an adult subject and/or a pediatric subject).

In still yet further embodiments of the above method, the biological sample is the sample is selected from the group consisting of a whole blood sample, a urine sample, a cerebrospinal fluid sample, a tissue sample, a bodily fluid sample, a saliva sample, an oropharyngeal specimen, and a nasopharyngeal specimen. For example, in some embodiments, the sample is a whole blood sample. In other embodiments, the sample is a plasma sample. In still further embodiments, the sample is a serum sample. In yet still further embodiments, the sample is a saliva sample. In still yet further embodiments, the sample is urine sample. In still yet other embodiments, the sample is an oropharyngeal sample. In yet other embodiments embodiments, the sample is a whole blood sample and the subject is a human subject (e.g., an adult and/or pediatric subject). In other embodiments, the sample is a plasma sample and the subject is a human subject (e.g., an adult and/or pediatric subject). In still further embodiments, the sample is a serum sample and the subject is a human subject (e.g., an adult and/or pediatric subject). In yet still further embodiments, the sample is a saliva sample and the subject is a human subject (e.g., an adult and/or pediatric subject). In still yet further embodiments, the sample is urine sample and the subject is a human subject (e.g., an adult and/or pediatric subject). In still yet other embodiments, the sample is an oropharyngeal sample and the subject is a human subject (e.g., an adult and/or pediatric subject).

In yet further embodiments of the above method, the at least one lateral flow assay for GFAP, the at least one lateral flow assay for UCH-L1, or at least one lateral flow assay for GFAP and at least one lateral flow assay for UCH-L1 are each performed or capable of being performed in less than about 30 minutes, less than about 25 minutes, less than about 20 minutes, less than about 18 minutes, or less than about 15 minutes. In yet still further embodiments of the above method, the at least one lateral flow assay for GFAP, the at least one lateral flow assay for UCH-L1, or at least one lateral flow assay for GFAP and at least one lateral flow assay for UCH-L1 are each are each performed or capable of being performed in a time ranging from about 4 to about 20 minutes, from about 10 to about 15 minutes or from about 15 to about 18 minutes.

In another embodiment, the present disclosure relates to a kit for performing the above method. The kit can comprise: (a) a first lateral flow device for detecting a presence of ubiquitin carboxy-terminal hydrolase L1 (UCH-L1) in the sample; (b) a second lateral flow device for detecting a presence of glial fibrillary acidic protein (GFAP) in the sample; and (c) instructions for detecting the presence of UCH-L1 and GFAP in the sample.

In another embodiment, the present disclosure relates to a kit for performing the above method. The kit can comprise: (a) a lateral flow device for detecting a presence of ubiquitin carboxy-terminal hydrolase L1 (UCH-L1) and glial fibrillary acidic protein (GFAP) in the sample; and (b) instructions for detecting the presence of UCH-L1 and GFAP in the sample. The lateral flow device contained in the kit can contain one test strip for detecting the presence or amount of UCH-L1 and GFAP in the sample. Alternatively, the lateral flow device can contain a first test strip for determining the presence or amount of UCH-L1 in the sample and a second test strip for determining the presence or amount of GFAP in the sample.

### DETAILED DESCRIPTION

The present disclosure relates to methods, lateral flow assays, and devices for determining the presence or amount of GFAP, UCH-L1, or GFAP and UCH-L1 is a sample obtained from a subject. In some embodiments, the methods involve detecting the presence of GFAP, UCH-L1, or GFAP and UCH-L1 in a sample obtained from a subject by performing a lateral flow assay. In other embodiments, the methods involve determining whether the level of GFAP, UCH-L1, or GFAP and UCH-L1 in a sample obtained from a subject is elevated by performing a lateral flow assay.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.”

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

An “amount” as used herein refers to a quantity specified (e.g., high or low) or a number e.g., where the number is a level, such as a position on a real or imaginary scale of amount or quantity, or a concentration, such as, for example, a relative amount of a given substance contained within a solution or in a particular volume of space, e.g., the amount of solute per unit volume of solution.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-UCH-L1 antibody or a UCH-L1 antibody).

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

“CDR” is used herein to refer to the “complementarity determining region” within about an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Control zone” or “control line” as used interchangeably herein refers a region of a test strip in which a label can be observed to shift location, appear, change color, or disappear to indicate that an assay performed correctly. Detection or observation of the control zone (e.g., of a control line) may be done by any convenient means, depending upon the particular choice of label, especially, for example but not limited to, visually, fluorescently, by reflectance, radiographically, and the like. In some embodiments, the label may or may not be applied directly to the control zone, depending upon the design of the control being used.

“CT scan” as used herein refers to a computerized tomography (CT) scan. A CT scan combines a series of X-ray images taken from different angles and uses computer processing to create cross-sectional images, or slices, of the bones, blood vessels and soft tissues inside your body. The CT scan may use X-ray CT, positron emission tomography (PET), single-photon emission computed tomography (SPECT), computed axial tomography (CAT scan), or computer aided tomography. The CT scan may be a conventional CT scan or a spiral/helical CT scan. In a conventional CT scan, the scan is taken slice by slice and after each slice the scan stops and moves down to the next slice, e.g., from the top of the abdomen down to the pelvis. The conventional CT scan requires patients to hold their breath to avoid movement artefact. The spiral/helical CT scan is a continuous scan which is taken in a spiral fashion and is a much quicker process where the scanned images are contiguous.

“Decentralize”, “Decentralized”, or “Decentralization”, as used interchangeably herein, refers to, in the context of testing, the performance of one or more medical tests and/or assays outside of a traditional medical setting (e.g., a hospital, physician office, stand alone lab site, etc.) to one or more places such as urgent care clinics, retail clinics, pharmacies, grocery stores or convenience stores, residences (e.g., homes, apartments, etc.), workplaces, and/or government offices (e.g., U.S. Transportation and Safety Authority), etc. “Hybrid-decentralization” or “hybrid-decentralized” refers to situations in which a subject or patient collects a sample at a residence and/or workplace and ships the sample to a laboratory, avoiding a professional collection site (such as a hospital, physician's office, or stand alone sample collection or lab site).

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25: 1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of UCH-L1, GFAP, or UCH-L1 and GFAP. Non-limiting examples of a DVD-Ig binding protein include (1) a DVD-Ig binding protein that binds one or more epitopes of UCH-L1, a DVD-Ig binding protein that binds an epitope of a human UCH-L1 and an epitope of UCH-L1 of another species (for example, mouse), and a DVD-Ig binding protein that binds an epitope of a human UCH-L1 and an epitope of another target molecule; (2) a DVD-Ig binding protein that binds one or more epitopes of GFAP, a DVD-Ig binding protein that binds an epitope of a human GFAP and an epitope of GFAP of another species (for example, mouse), and a DVD-Ig binding protein that binds an epitope of a human GFAP and an epitope of another target molecule; or (3) a DVD-Ig binding protein that binds one or more epitopes of UCH-L1 and GFAP, a DVD-Ig binding protein that binds an epitope of a human UCH-L1, a human GFAP, and an epitope of UCH-L1 of another species (for example, mouse), and a DVD-Ig binding protein that binds an epitope of a human UCH-L1, a human GFAP, and an epitope of another target molecule.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“GFAP” is used herein to describe glial fibrillary acidic protein. GFAP is a protein that is encoded by the GFAP gene in humans and by GFAP gene counterparts in other species, and which can be produced (e.g., by recombinant means, in other species).

“GFAP status” can mean either the level or amount of GFAP at a point in time (such as with a single measure of GFAP), the level or amount of GFAP associated with monitoring (such as with a repeat test on a subject to identify an increase or decrease in GFAP amount), the level or amount of GFAP associated with treatment for traumatic brain injury (whether a primary brain injury and/or a secondary brain injury) or combinations thereof.

“Glasgow Coma Scale” or “GCS” as used herein refers to a 15 point scale (e.g., described in 1974 by Graham Teasdale and Bryan Jennett, Lancet 1974; 2:81-4) that provides a practical method for assessing impairment of conscious level in patients who have suffered a brain injury. The test measures the best motor response, verbal response and eye opening response with these values: I. Best Motor Response (6—obey 2-part request; 5—brings hand above clavicle to stimulus on head neck; 4—bends arm at elbow rapidly but features not predominantly abnormal; 3—bends arm at elbow, features clearly predominantly abnormal; 2—extends arm at elbow; 1—no movement in arms/legs, no interfering factor; NT—paralyzed or other limiting factor); II. Verbal Response (5—correctly gives name, place and date; 4—not orientated but communication coherently; 3—intelligible single words; 2—only moans/groans; 1—no audible response, no interfering factor; NT—factor interfering with communication); and III. Eye Opening (4—open before stimulus; 3—after spoken or shouted request; 2—after fingertip stimulus; 1—no opening at any time, no interfering factor; NT—closed by local factor). The final score is determined by adding the values of I+II+III. A subject is considered to have a mild TBI if the GCS score is 13-15. A subject is considered to have a moderate TBI if the GCS score is 9-12. A subject is considered to have a severe TBI if the GCS score is 8 or less, typically 3-8.

“Glasgow Outcome Scale” as used herein refers to a global scale for functional outcome that rates patient status into one of five categories: Dead, Vegetative State, Severe Disability, Moderate Disability or Good Recovery. “Extended Glasgow Outcome Scale” or “GOSE” as used interchangeably herein provides more detailed categorization into eight categories by subdividing the categories of severe disability, moderate disability and good recovery into a lower and upper category as shown in Table 1.

TABLE 1

| | | | |
|---|---|---|---|
| 1 | Death | D | |
| 2 | Vegetative | VX | |
| | state | | |
| 3 | Lower severe | SD− | Condition of unawareness with only reflex |
| | disability | | responses but with periods of spontaneous |
| 4 | Upper severe | SD+ | eye opening. If the patient can be left |
| | disability | | alone for more than 8 hours at home it is |
| | | | upper level of SD, if not then it is low |
| | | | level of SD. |
| 5 | Lower moderate | MD− | Patient who is dependent for daily support |
| | disability | | for mental or physical disability, usually a |
| 6 | Upper moderate | MD+ | combination of both. If they are able to |
| | disability | | return to work even with special |
| | | | arrangement it is upper level of MD, if not |
| | | | then it is low level of MD. |
| 7 | Lower good | GR− | Patients have some disability such as |
| | recovery | | aphasia, hemiparesis or epilepsy and/or |
| 8 | Upper good | GR+ | deficits of memory or personality but are |
| | recovery | | able to look after themselves. They are |
| | | | independent at home but dependent |
| | | | outside. |

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

The term “immunoassay,” as used herein, refers to a biochemical test that measures the presence or concentration of a macromolecule or a small molecule in a solution through the use of an antibody or an antigen. Any suitable immunoassay may be used, and a wide variety of immunoassay types, configurations, and formats are known in the art and within the scope of the present disclosure. Suitable types of immunoassays include, but are not limited to, enzyme-linked immunosorbent assay (ELISA), lateral flow assay, competitive inhibition immunoassay (e.g., forward and reverse), radioimmunoassay (RIA), fluoroimmunoassay (FIA), chemiluminescent immunoassay (CLIA), counting immunoassay (CIA), enzyme multiplied immunoassay technique (EMIT), one-step antibody detection assay, homogeneous assay, heterogeneous assay, capture on the fly assay, single molecule detection assay, etc. Such methods are disclosed in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792; International Patent Application Publications WO 2016/161402 and WO 2016/161400; and Adamczyk et al., Anal. Chim. Acta, 579(1): 61-67 (2006).

The immunoassay format may be “direct” or “indirect” “sandwich.” Sandwich formats involve the use of capture and detection antigens to immobilize and detect an antigen in a sample. Specifically, the surface of a solid support (e.g., ELISA plate, bead, etc.) is coated with a capture antibody or antigen-binding fragment thereof, which capture antibody binds to and immobilizes a target antigen present in a sample applied thereto. A detection antibody is then added or contacted to the complex. The detection antibody can be directly labeled with an antibody (“direct sandwich immunoassay”) to allow for detection and quantification of the antigen. Alternatively, if the detection antibody is unlabeled, a secondary enzyme-conjugated detection antibody may be used (“indirect sandwich assay”).

Thus, the disclosed method may further comprise contacting the sample with a conjugate comprising a second antibody, wherein second antibody, or antigen-binding fragment thereof, portion of the conjugate specifically binds to a target antigen (e.g., a protein from the GFAP, UCH-L1 or a fragment or epitope thereof), which results in the linkage of the conjugate to the captured analyte and formation of an immunosandwich (also referred to herein as an “immunosandwich complex”). It will be appreciated that, in sandwich immunoassay formats, the first antibody and the second antibody recognize two different non-overlapping epitopes on a target analyte/antigen.

As used herein, the term “immunochromatographic test(s)” (ICT) refers to an assay or test that comprises a cartridge(s) or strip(s) (typically single-use and/or disposable) which generates a detectable (e.g., such as colored) end product that can be interpreted as positive or negative. Immunochromatographic tests typically rely on the capture of a target analyte (e.g., antigen and/or antibody) from a biological sample. The assay or test utilizes a first specific binding member (e.g., antigen and/or antibody) mounted on a test strip as the immobilized capture specific binding member (test area). Capillary flow is used to move a detectably-labeled second specific binding member conjugate which binds to the target analyte in the mobile phase as it moves toward the capture first specific binding member in the immobile phase. A positive test is produced by the capture of the moving labeled second specific binding member complex by the first immobilized specific binding in the test area, and the formation of a colored line or pattern. One example of an ICT is a lateral flow assay.

“Injury to the head” or “head injury” as used interchangeably herein, refers to any trauma to the scalp, skull, or brain. Such injuries may include only a minor bump on the head or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), a cerebral vascular accident (e.g., stroke), one or more falls (e.g., as in sports or other activities), explosions or blasts (collectively, “blast injuries”) and by other types of blunt force trauma. Alternatively, an injury to the head may be caused by the ingestion and/or exposure to a chemical, toxin or a combination of a chemical and toxin. Examples of such chemicals and/or toxins include fires, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, hypoxia, a viral infection (e.g., SARS-CoV-2), a fungal infection, a bacterial infection, meningitis, hydrocephalus, or any combinations thereof. In some cases, it is not possible to be certain whether any such event or injury has occurred or taken place. For example, there may be no history on a patient or subject, the subject may be unable to speak, the subject may be aware of what events they were exposed to, etc. Such circumstances are described herein as the subject “may have sustained an injury to the head,” or as a “suspected injury”. In certain embodiments herein, the closed head injury does not include and specifically excludes a cerebral vascular accident, such as stroke.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling. Any suitable detectable label known in the art can be used.

A “lateral flow device” or “test device” as used interchangeably herein, refers to a device that allows for the lateral flow detection of a biomarker immobilized on a substrate (e.g., test strip, a membrane, an absorbent pad (e.g., wicking pad), etc.) using a specific binding agent. Described herein are exemplary lateral flow devices and methods of using such devices that allow for efficient lateral flow detection of GFAP, UCH-L1 and/or GFAP and UCH-L1 immobilized on a substrate using specific binding agents (e.g., anti-GFAP antibodies, anti-UCH-L1 antibodies or anti-GFAP and anti-UCH-L1 antibodies).

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6× His tag, which has an amino acid sequence of HHHHHH (SEQ ID NO:3), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO:4) and derivatives thereof (e.g., ADDDDK (SEQ ID NO:5), etc.; (iii) Miscellaneous sequences can be used to link or connect the light and/or heavy chain variable regions of single chain variable region fragments. Examples of other linking sequences can be found in Bird et al., Science 242: 423-426 (1988); Huston et al., PNAS USA 85: 5879-5883 (1988); and McCafferty et al., Nature 348: 552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen (e.g., although cross-reactivity or shared reactivity may occur). Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Magnetic resonance imaging” or “MRI” as used interchangeably herein refers to a medical imaging technique used in radiology to form pictures of the anatomy and the physiological processes of the body in both health and disease (e.g., referred to herein interchangeably as “an MRI”, “an MRI procedure” or “an MRI scan”). MRI is a form of medical imaging that measures the response of the atomic nuclei of body tissues to high-frequency radio waves when placed in a strong magnetic field, and that produces images of the internal organs. MRI scanners, which is based on the science of nuclear magnetic resonance (NMR), use strong magnetic fields, radio waves, and field gradients to generate images of the inside of the body.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Proximal end” refers as used herein refers to the end of a test device or test strip that includes the sample application aperture of the test device and/or the sample application zone of the test strip.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multivalent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Result” as used herein refers to an item of information obtained by performing an assay. In one embodiment, a result is an amount of a biomarker (e.g., UCH-L1, GFAP, or any combination thereof) in a test sample. In another embodiment, a result is identifying the presence of biomarker (e.g., UCH-L1, GFAP, or any combination thereof) in a sample. In some embodiments, a result is displayed as a numerical value e.g., done with the use of a reading device or reader. In other embodiments, a result is visually displayed (e.g., as a colored line).

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” and “patient sample” as used herein may be used interchangeable and may be a sample of blood, such as whole blood (including for example, capillary blood, venous blood, a mixed sample of venous and capillary blood, a mixed sample of capillary blood and interstitial fluid, dried blood spot, etc.), tissue, urine, semen, serum, plasma, saliva, sweat, sputum, mucus, lacrimal fluid, lymph fluid, amniotic fluid, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, nasal mucus specimens, frozen sections taken for histologic purposes, blood (such as whole blood, capillary blood, venous blood, a mixed sample of venous and capillary blood, a mixed sample of capillary blood and interstitial fluid, dried blood spots, etc.), plasma, serum, red blood cells, platelets, an anal sample (such as an anal swab specimen), interstitial fluid, cerebrospinal fluid, etc. Cell types and tissues may also include lymph fluid, cerebrospinal fluid, or any fluid collected by aspiration. A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some embodiments, the sample is a whole blood sample. In some embodiments, the sample is a capillary blood sample. In some embodiments, the sample is a dried blood spot. In some embodiments, the sample is a serum sample. In yet other embodiments, the sample is a plasma sample. In some embodiments, the sample is an oropharyngeal specimen. In other embodiments, the sample is a nasopharyngeal specimen. In other embodiments, the sample is sputum. In other embodiments, the sample is endotracheal aspirate. In still yet other embodiments, the sample is bronchoalveolar lavage. In still yet other embodiments, the sample is mucus. In still yet other embodiments, the sample is saliva. In still further embodiments, the sample is urine.

“Sample application aperture” as used herein refers to the portion of a test device where an opening in the test device provides access to the sample application zone of the test strip.

“Sample application zone” is the portion of a test strip where sample is applied. In some embodiments, a “sample pad” comprises a sample application zone.

“Sensitivity” refers to the proportion of subjects for whom the outcome is positive that are correctly identified as positive (e.g., correctly identifying those subjects with a disease or medical condition for which they are being tested). For example, this might include correctly identifying subjects as having a TBI as distinct from those who do not have a TBI, correctly identifying subjects having a moderate, severe, or moderate to severe TBI as distinct from those having a mild TBI, correctly identifying subjects as having a mild TBI as distinct from those having a moderate, severe, or moderate to severe TBI, correctly identifying subjects as having a moderate, severe, or moderate to severe TBI as distinct from those having no TBI or correctly identifying subjects as having a mild TBI from those having no TBI, etc.).

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects having an TBI as distinct from those who do not have a TBI, correctly identifying subjects not having a moderate, severe, or moderate to severe TBI as distinct from those having a mild TBI, correctly identifying subjects as not having a mild TBI as distinct from those having a moderate, severe, or moderate to severe TBI or correctly identifying subjects as not having any TBI, or correctly identifying subjects as having a mild TBI as distinct from those having no TBI, etc.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In some embodiments, the subject is a human. The subject or patient may be undergoing other forms of treatment. In some embodiments, the subject is a human that may be undergoing other forms of treatment. In some embodiments the subject is a human-helper subject—e.g., a horse, dog, or other species that assists humans in carrying out their daily tasks (e.g., companion animal) or occupation (e.g., service animal).

“Test strip” as used herein can include one or more bibulous or non-bibulous materials. If a test strip comprises more than one material, the one or more materials are preferably in fluid communication. One material of a test strip may be overlaid on another material of the test strip, such as for example, filter paper overlaid on nitrocellulose. Alternatively or additionally, a test strip may include a region comprising one or more materials followed by a region comprising one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, polyethylene terephthalate (e.g., DACRON brand polymer), PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH, salt concentration, or specific gravity of an applied sample to optimize test conditions.

The material or materials can be a single structure such as a sheet cut into strips or it can be several strips or particulate material bound to a support or solid surface such as found, for example, in thin-layer chromatography and may have an absorbent pad either as an integral part or in liquid contact. The material can also be a sheet having lanes thereon, capable of spotting to induce lane formation, wherein a separate assay can be conducted in each lane. The material can have a rectangular, circular, oval, triangular, or other shape provided that there is at least one direction of traversal of a test solution by capillary migration. Other directions of traversal may occur such as in an oval or circular piece contacted in the center with the test solution. However, the main consideration is that there be at least one direction of flow to a predetermined site.

The support for the test strip, where a support is desired or necessary, will normally be water insoluble, frequently non-porous and rigid but may be elastic, usually hydrophobic, and porous and usually will be of the same length and width as the strip but may be larger or smaller. The support material can be transparent, and, when a test device of the present disclosure is assembled, a transparent support material can be on the side of the test strip that can be viewed by the user, such that the transparent support material forms a protective layer over the test strip where it may be exposed to the external environment, such as by an aperture in the front of a test device. A wide variety of non-mobilizable and non-mobilizable materials, both natural and synthetic, and combinations thereof, may be employed provided only that the support does not interfere with the capillary action of the material or materials, or non-specifically bind assay components, or interfere with the signal producing system. Illustrative polymers include polyethylene, polypropylene, poly(4-methylbutene), polystyrene, polymethacrylate, poly(ethylene terephthalate), nylon, poly(vinyl butyrate), glass, ceramics, metals, and the like. Elastic supports may be made of polyurethane, neoprene, latex, silicone rubber and the like.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” “moderate to severe”, or “severe.” The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more fires, chemicals or toxins (such as molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, a viral infection (e.g., SARS-CoV-2, meningitis, etc.), a fungal infection, a bacterial infection (e.g., meningitis), hydrocephalus, or any combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a head injury where a subject may or may not experience a loss of consciousness. For subjects that experience a loss of consciousness, it is typically brief, usually lasting only a few seconds or minutes. Mild TBI is also referred to as a concussion, minor head trauma, minor TBI, minor brain injury, and minor head injury. While MRI and CT scans are often normal, the individual with mild TBI may have cognitive problems such as headache, difficulty thinking, memory problems, attention deficits, mood swings and frustration.

Mild TBI is the most prevalent TBI and is often missed at time of initial injury. Typically, a subject has a Glasgow Coma Scale score of between 13-15 (such as 13-15 or 14-15). Fifteen percent (15%) of people with mild TBI have symptoms that last 3 months or more. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Moderate TBI” as used herein refers to a brain injury where loss of consciousness and/or confusion and disorientation is between 1 and 24 hours and the subject has a Glasgow Coma Scale score of between 9-13 (such as 9-12 or 9-13). The individual with moderate TBI may have abnormal brain imaging results.

“Severe TBI” as used herein refers to a brain injury where loss of consciousness is more than 24 hours and memory loss after the injury or penetrating skull injury longer than 24 hours and the subject has a Glasgow Coma Scale score between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

“Moderate to severe” TBI as used herein refers to a spectrum of brain injury that includes a change from moderate to severe TBI over time and thus encompasses (e.g., temporally) moderate TBI alone, severe TBI alone, and moderate to severe TBI combined. For example, in some clinical situations, a subject may initially be diagnosed as having a moderate TBI but who, over the course of time (minutes, hours or days), progresses to having a severe TBI (such, as for example, in situations when there is a brain bleed). Alternatively, in some clinical situations, a subject may initially be diagnosed as having a severe TBI but who, over the course of time (minutes, hours or days), progresses to having a moderate TBI. Such subjects would be examples of patients that could be classified as “moderate to severe”. Common symptoms of moderate to severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing issues, such as decrease or loss of hearing, ringing in the ears (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, problems with control of bowel and bladder, sleep disorders, loss of stamina, appetite changes, problems with regulation of body temperature, menstrual difficulties, dependent behaviors, issues with emotional ability or stability, lack of motivation, irritability, aggression, depression, disinhibition, or denial/lack of awareness. Subjects having a moderate to severe TBI can have a Glasgow Coma Scale score from 3-12 (which includes the range of 9-12 for a moderate TBI, and 3-8 for a severe TBI).

“Ubiquitin carboxy-terminal hydrolase L1” or “UCH-L1” as used interchangeably herein refers to a deubiquitinating enzyme encoded by the UCH-L1 gene in humans and by UCH-L1 gene counterparts in other species. UCH-L1, also known as ubiquitin carboxyl-terminal esterase L1 and ubiquitin thiolesterase, is a member of a gene family whose products hydrolyze small C-terminal adducts of ubiquitin to generate the ubiquitin monomer.

“UCH-L1 status” can mean either the level or amount of UCH-L1 at a point in time (such as with a single measure of UCH-L1), the level or amount of UCH-L1 associated with monitoring (such as with a repeat test on a subject to identify an increase or decrease in UCH-L1 amount), the level or amount of UCH-L1 associated with treatment for traumatic brain injury (whether a primary brain injury and/or a secondary brain injury) or combinations thereof.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one embodiment, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-UCH-L1 antibody that differs from the corresponding fragment of anti-UCH-L1 antibody in amino acid sequence but is still antigenically reactive and can compete with the corresponding fragment of anti-UCH-L1 antibody for binding with UCH-L1. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

As used herein “zone” refers to a region on a test strip. In some embodiments, the zone can be a “reagent zone”. A “reagent zone” as used herein refers a region of a test strip where reagent is provided. The reagent zone can be on a reagent pad, a separate segment of bibulous or non-bibulous material included on the test strip, or it can be a region of a bibulous or non-bibulous material of a test strip that also includes other zones, such as an analyte detection zone. The reagent zone can carry a detectable label, which may be a direct or indirect label. Preferably the reagent is provided in a form that is immobile in the dry state and mobile in the moist state. A reagent can be a specific binding member, an analyte or analyte analog, an enzyme, a substrate, indicators, components of a signal producing system, chemicals or compounds such as buffering agents, reducing agents, chelators, surfactants, etc., that contribute to the function of the test strip assay.

In other embodiments, the zone can be a test results zone. A “test results zone” used herein refers to a region of a test strip that provides a detectable signal indicating the presence of the analyte. The test results zone can include an immobilized binding reagent specific for an analyte (“specific binding member”) and/or an enzyme that reacts with the analyte. A test results zone can include one or more analyte detection zones, e.g., a “test line”. Other substances that may allow or enhance detection of the analyte, such as substrates, buffers, salts, may also be provided in the test results zone. One or more members of a signal producing system may be bound directly or indirectly to the detection zone. A test results zone can optionally include one or more control zones (e.g., a “control line”) that provide indication that the test has been performed properly.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Lateral Flow Assays, Devices, and Methods for Detecting the Presence or Amount of GFAP, UCH-L1 or Both UCH-L1 and GFAP in Samples Obtained from Subjects

In one embodiment, the present disclosure relates to lateral flow assays. Lateral flow assays are generally provided as part of a lateral flow device or test device comprising a lateral flow test strip (e.g., nitrocellulose or filter paper), a sample application area (e.g., sample pad), a test results area (e.g., where the results are displayed (e.g., as a test line or as a numerical value)), an optional control results area (e.g., a control line), and an analyte-specific binding partner that is bound to a detectable label (e.g., a colored particle or an enzyme detection system). See, e.g., U.S. Pat. Nos. 6,485,982; 6,187,598; 5,622,871; 6,548,309; 6,565,808; and 6,809,687 and U.S. Patent Publication No. 2004/0184954, each of which is incorporated herein by reference.

In some embodiments, the lateral flow assay is performed to determine the presence (e.g., a qualitative determination) or amount (e.g., a quantitative determination) of GFAP in a sample (e.g., such as by the use of at least one specific binding partner, such as an anti-GFAP antibody). For example, the lateral flow assay can be used to determine the presence or amount of GFAP in the sample by using at least one specific binding partner which specifically binds to an epitope on GFAP and a second specific binding partner that comprises a detectable label and that specifically binds to a different epitope on GFAP than the first specific binding partner. In other embodiments, the lateral flow assay can be used to determine the presence (e.g., a qualitative determination) or amount (e.g., a quantitative determination) UCH-L1 in a sample (e.g., such as by using at least one specific binding partner, such as an anti-UCH-L1 antibody). For example, the lateral flow assay determines the presence or amount of UCH-L1 in the sample by using at least one specific binding partner which specifically binds to an epitope on UCH-L1 and a second specific binding partner that comprises a detectable label and specifically binds to a different epitope on UCH-L1 than the first specific binding partner. In yet further embodiments, a lateral flow assay is used to determine the presence or amount of each of GFAP and UCH-L1 in the sample. In some embodiments, at least two separate lateral flow assays are used to determine the presence or amount of GFAP and UCH-L1 in a sample (e.g., such as by the use of at least one first specific binding partner for GFAP, such as an anti-GFAP antibody, and at least one second specific binding partner for UCH-L1, such as an anti-UCH-L1 antibody). If at least two separate lateral flow assays are used to determine the presence or amount of GFAP and UCH-L1 in a sample, the assays can be performed simultaneously or sequentially, in any order. In other embodiments, a single lateral flow assay can be used to determine the presence or amount of GFAP and UCH-L1 in a sample. For example, a single lateral flow assay can be used to determine the presence or amount at least one epitope on GFAP (e.g., such as by use of at least one first specific binding partner for GFAP) and at least one epitope on UCH-L1 (e.g., such as by use of at least one second specific binding partner for UCH-L1) in the sample as described previously herein. In yet other embodiments, the lateral flow assay detects both GFAP and UCH-L1 in a sample.

In some embodiments, the disclosure relates to a test device comprising reagent-impregnated test strips to provide a specific binding assay, e.g., an immunoassay. In some embodiments, a sample is applied to one portion of the test strip and is allowed to permeate through the strip material, usually with the aid of an eluting solvent such as water and/or a suitable buffer. In additional embodiments, the strip material can further comprise a detergent. The sample progresses into or through a detection zone in the test strip wherein at least one specific binding partner (e.g., at least one antibody) for an analyte (e.g., GFAP, UCH-L1, or GFAP and UCH-L1) or a fragment or variant thereof, suspected of being in the sample is immobilized. Any analyte (e.g., GFAP, UCH-L1, or GFAP and UCH-L1) present in the sample can become bound within the detection zone. The extent to which the analyte (e.g., GFAP, UCH-L1, or GFAP and UCH-L1) becomes bound in that zone can be determined with the aid of labelled reagents (e.g., specific binding partners labeled with a detectable label, such as for example, a labeled anti-GFAP antibody and/or a labeled anti-UCH-L1 antibody) that can also be incorporated in the test strip or applied thereto subsequently. More specifically, in some embodiments, the lateral flow device comprises a single strip which contains a specific binding partner (e.g., at least one antibody) for UCH-L1 and at least one specific binding partner (e.g., at least one antibody) for GFAP. Any UCH-L1 and/or GFAP present in the sample can become bound within the detection zone in the test strip as discussed previously herein. In other embodiments, the lateral flow device comprises at least two single strips, a first strip which contains a specific binding partner (e.g., at least one antibody) for UCH-L1 and a second strip which contains a specific binding partner (e.g., at least one antibody) for GFAP. Any UCH-L1 and/or GFAP present in the sample can become bound within the detection zone in each respective strip.

In some embodiments, the analytical test device comprises a hollow casing constructed of moisture-impervious solid material containing a dry porous carrier that communicates directly or indirectly with the exterior of the casing such that a liquid test sample can be applied to the porous carrier. In some embodiments, the device also comprises a labelled specific binding partner for an analyte and the labelled specific binding partner is freely mobile within the porous carrier when in the moist state. In some embodiments, the device comprises unlabeled specific binding partner for the same analyte and the unlabeled reagent is permanently immobilized in a detection zone on the carrier material and is therefore not mobile in the moist state. The relative positioning of the labelled reagent and detection zone being such that liquid sample applied to the test device can pick up labelled reagent and thereafter permeate into the detection zone and the test device provides the extent (if any) to which the labelled reagent becomes in the detection zone to be observed.

Another embodiment of the disclosure relates to a test device that comprises a porous solid phase material carrying in a first zone a labelled reagent that is retained in the first zone while the porous material is in the dry state but is free to migrate through the porous material when the porous material is moistened, for example, by the application of an aqueous liquid sample suspected of containing the analyte. In some embodiments, the porous material comprises in a second zone, which is spatially distinct from the first zone, an unlabeled specific binding partner having specificity for the analyte and which is capable of participating with the labelled reagent in either a “sandwich” or a “competition” reaction. The unlabeled specific binding partner is firmly immobilized on the porous material such that it is not free to migrate when the porous material is in the moist state.

In other embodiments, the disclosure also provides a method in which a test device as described herein is contacted with an aqueous liquid sample suspected of containing the analyte, such that the sample permeates by capillary action through the porous solid phase material via the first zone into the second zone and the labelled reagent migrates therewith from the first zone to the second zone, the presence of analyte in the sample being determined by observing the extent (if any) to which the labelled reagent becomes bound in the second zone.

In some embodiments, the labelled reagent is a specific binding partner for the analyte (e.g., GFAP and/or UCH-L1). The labelled reagent, the analyte (if present), and the immobilized unlabeled specific binding partner cooperate together in a “sandwich” reaction. This results in the labelled reagent being bound in the second zone if analyte is present in the sample. In a sandwich format, the two binding reagents have specificities for different epitopes (e.g., specifically bind to different epitopes) on the analyte.

In some embodiments, the labelled reagent is an antibody to analyte (e.g., an anti-GFAP antibody labeled with a detectable label and/or anti-UCH-L1 antibody labeled with a detectable label) the analyte itself (e.g., GFAP conjugated with a detectable label and/or UCH-L1 conjugated with a detectable label) or a fragment or variant thereof. In some embodiments, the labelled antibody or labelled analyte or fragment or variant thereof migrates through the porous solid phase material into the second zone and binds with the immobilized reagent. An analyte (e.g., GFAP and/or UCH-L1) present in the sample competes with the labelled reagent in this binding reaction. Such competition results in a reduction in the amount of labelled reagent binding in the second zone and a consequent decrease in the intensity of the signal observed in the second zone in comparison with the signal that is observed in the absence of analyte in the sample.

In some embodiments, a test strip (e.g., the carrier material) comprises nitrocellulose. This has considerable advantage over some other strip materials, such as paper, because it has a natural ability to bind proteins without requiring prior sensitization. Specific binding partners, such as antibodies (such as an anti-GFAP antibody, anti-UCH-L1 antibody or anti-GFAP antibody and anti-UCH-L1 antibody), can be applied directly to nitrocellulose and immobilized thereon. No chemical treatment is required that might interfere with the essential specific binding activity of the reagent. Unused binding sites on the nitrocellulose can thereafter be blocked using simple materials, such as polyvinylalcohol. Moreover, nitrocellulose is readily available in a range of pore sizes and this facilitates the selection of a carrier material to suit particularly requirements such as sample flow rate.

In some embodiments, the disclosure comprises the use of one or more direct labels attached to one of the specific binding partners. In some embodiments, the technology uses a label comprising, e.g., colloidal metal (e.g., a sol or colloidal suspension of gold or silver particles (e.g., gold nanoparticles, silver nanoparticles, etc.), colloidal non-metal (e.g., a sol or colloidal suspension of selenium or tellurium particles) in a fluid, usually water or an aqueous buffer), a color or dye (e.g., a dye sol), a latex particle (including a colored or non-colored latex particle), or any combinations thereof. In some embodiments, a label produces an instant analytical result without the need to add further reagents to develop a detectable signal. Additionally, such label is visible to the naked eye (e.g., does not require the use of a device to read the amount or presence of UCH-L1, GFAP, or UCH-L1 and GFAP (e.g., a reader or reading device). They are robust and stable and can therefore be used readily in an analytical test device which is stored in the dry state. Their release on contact with an aqueous sample can be modulated, for example, by the use of soluble glazes.

In some embodiments, development of the test devices described herein involves the selection of technical features that enable a direct labelled specific binding partner to be used in a carrier-based analytical test device, e.g. one based on a strip format, to give a quick and clear result. In some embodiments, the result of the assay are displayed visually (e.g., discernable by eye) and to facilitate this, it is necessary for the direct label to become concentrated in the detection zone. To achieve this, the direct labelled reagent should be transportable easily and rapidly by the developing liquid. Furthermore, it is preferable that the whole of the developing sample liquid is directed through a comparatively small detection zone in order that the probability of an observable result being obtained in increased. In other embodiments, the results of the assay are displayed as a numerical value. In these embodiments, where the results are in the form of a numerical value, optionally a reader or reading device can be used, e.g., to produce the displayed value.

In some embodiments, the disclosure comprises use of a directly labelled specific binding partner on a carrier material comprising nitrocellulose. In some embodiments, the nitrocellulose has a pore size of at least about one micron. In some embodiments, the nitrocellulose has a pore size not greater than about 20 microns. In some embodiments, the direct label is a colored latex particle of spherical or near-spherical shape and having a maximum diameter of not greater than about 0.5 micron. In some embodiments, the size range for such particles is from about 0.05 to about 0.5 microns.

In some embodiments, the porous solid phase material is linked to a porous receiving member to which the liquid sample can be applied and from which the sample can permeate into the porous solid phase material. In some embodiments, the porous solid phase material is contained within a moisture-impermeable casing or housing and the porous receiving member, with which the porous solid phase material is linked, extends out of the housing and can act as a means for permitting a liquid sample to enter the housing and permeate the porous solid phase material. The housing should be provided with means, e.g., appropriately placed apertures, that enable the second zone of the porous solid phase material (carrying the immobilized unlabeled specific binding partner) to be observable from outside the housing so that the result of the assay can be observed. If desired, the housing may also be provided with further means which enable a further zone of the porous solid phase material to be observed from outside the housing and which further zone one incorporates control reagents which enable an indication to be given as to whether the assay procedure has been completed. In some embodiments, the housing is provided with a removable cap or shroud that can protect the protruding porous receiving member during storage before use. In some embodiments, if desired, the cap or shroud can be replaced over the protruding porous receiving member, after sample application, while the assay procedure is being performed. optionally, the labelled reagent can be incorporated elsewhere within the test device, e.g., in the bibulous sample collection member.

In some embodiments, test devices are provided as kits suitable for use in a hospital, or in a decentralized setting. For example, the test devices can be used in an urgent care clinic, a pharmacy, a grocery or other convenience store, a residence, a workplace, and/or a government office. In other embodiments, the test devices are provided as kits suitable for use by the end user (e.g., as a self-test). In some embodiments, kits comprise a plurality (e.g., two) of test devices individually wrapped in moisture impervious wrapping and packaged together with appropriate instructions to the user. For example, in this embodiment, the kit comprises a first lateral flow device for detecting the presence or amount of GFAP in a sample and/or a second lateral flow device for detecting the presence or amount of UCH-L1 in the sample. In other embodiments, the kits comprise a single test device individually wrapped in moisture impervious wrapping and packaged with appropriate instructions to the user. For example, in this embodiment, the kit comprises a lateral flow device containing one or more strips for detecting the presence or amount of GFAP and/or UCH-L1 in a sample.

In some embodiments, the test device comprises a porous sample receiving member. In some embodiments, the test device comprises a hollow elongated casing containing a dry porous nitrocellulose carrier that communicates indirectly with the exterior of the casing via a bibulous sample receiving member that protrudes from the casing. In some embodiments, a porous sample receiving member is made from any bibulous, porous, or fibrous material capable of absorbing liquid rapidly. The porosity of the material can be unidirectional (e.g., with pores or fibers running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile, and polytetrafluoro-ethylene can be used. It can be advantageous to pre-treat the member with a surface-active agent during manufacture, e.g., to reduce any inherent hydrophobicity in the member and therefore enhance its ability to take up and deliver a moist sample rapidly and efficiently. Porous sample receiving members can also be made from paper or other cellulosic materials, such as nitrocellulose. Materials that are now used in the nibs of so-called fiber tipped pens are particularly suitable and such materials can be shaped or extruded in a variety of lengths and cross-sections appropriate in the context of the invention. In some embodiments, the material comprising the porous receiving member is chosen such that the porous member can be saturated with aqueous liquid within a matter of seconds. Preferably the material remains robust when moist, and for this reason paper and similar materials are less preferred in any embodiment wherein the porous receiving member protrudes from a housing. The liquid must thereafter permeate freely from the porous sample receiving member into the porous solid phase material.

In some embodiments, the test device comprises an optional “control zone”. If present, the “control” zone can be designed to convey an unrelated signal to the user that the test device has worked. For example, the control zone can be loaded with an antibody (e.g., anti-rabbit IgG) that will bind to a labelled antibody from the first zone, e.g., a labeled rabbit IgG, to confirm that the sample has permeated the test strip. In some embodiments, the first zone comprises an antigen and/or antibody that is unrelated to the analyte (e.g., GFAP and/or UCH-L1) and that is specifically captured at the control zone. In some embodiments, the control zone can contain an anhydrous reagent that, when moistened, produces a color change or color formation, e.g. anhydrous copper sulphate which will turn blue when moistened by an aqueous sample. As an additional alternative, a control zone could contain immobilized analyte that reacts with excess labelled reagent from the first zone. As the purpose of the control zone is to indicate to the user that the test has been completed, the control zone should be located downstream from the second zone in which the desired test result is recorded. A positive control indicator therefore tells the user that the sample has permeated the required distance through the test device.

The label can be any entity the presence of which can be readily detected. In some embodiments, the label is a direct label, e.g., an entity that, in its natural state, is readily visible either to the naked eye or with the aid of an optical filter and/or applied stimulation, e.g., UV light to promote fluorescence. For example, minute colored particles, such as dye sols, metallic sols (e.g. gold), and colored latex particles, are very suitable. Concentration of the label into a small zone or volume gives rise to a readily detectable signal, e.g., a strongly-colored area. This can be evaluated by eye, or by instruments if desired.

In other embodiments, the disclosure comprises use of an indirect label. Indirect labels, such as enzymes, e.g., alkaline phosphatase and horseradish peroxidase, can be used but these usually require the addition of one or more developing reagents such as substrates before a visible signal can be detected. Such additional reagents can be incorporated in the porous solid phase material or in the sample receiving member, if present, such that they dissolve or disperse in the aqueous liquid sample. Alternatively, the developing reagents can be added to the sample before contact with the porous material or the porous material can be exposed to the developing reagents after the binding reaction has taken place.

Coupling of the label to a specific binding partner can be by covalent bonding, if desired, or by hydrophobic bonding.

In some embodiments, the labelled reagent migrates with the liquid sample as it progresses to the detection zone. In other embodiments, the flow of sample continues beyond the detection zone and sufficient sample is applied to the porous material so that this may occur and that any excess labelled reagent from the first zone that does not participate in any binding reaction in the second zone is flushed away from the detection zone by this continuing flow. If desired, an absorbent “sink” can be provided at the distal end of the carrier material. For example, the absorbent sink may comprise of, for example, Whatman 3 MM chromatography paper, to provide sufficient absorptive capacity to allow any unbound conjugate to wash out of the detection zone. As an alternative to such a sink, it can be sufficient to have a length of porous solid phase material which extends beyond the detection zone.

In some embodiments, the presence or intensity of the signal from the label that becomes bound in the second zone provides a qualitative (e.g., presence) or quantitative (e.g., amount) measurement of GFAP, UCH-L1, or GFAP and UCH-L1 in the sample. A plurality of detection zones arranged in series on the porous solid phase material, through which the aqueous liquid sample can pass progressively, can also be used to provide a quantitative measurement of the GFAP, UCH-L1, or GFAP and UCH-L1, or can be loaded individually with different specific binding agents to provide a multi-analyte test.

In some embodiments, the immobilized specific binding partner in the second zone is an antibody (e.g., a monoclonal antibody) that specifically binds to GFAP, UCH-L1, or GFAP and UCH-L1. In the embodiment of the technology involving the sandwich reaction, the labelled reagent is also an antibody (e.g., a monoclonal antibody) that specifically binds to GFAP, UCH-L1 or GFAP and UCH-L1. The immobilized antibody and the labeled antibody should each bind to different epitopes on GFAP, UCH-L1 or GFAP and UCH-L1.

In some embodiments, the carrier material is in the form of a strip or sheet to which the reagents are applied in spatially distinct zones and the liquid sample is allowed to permeate through the sheet or strip from one side or end to another.

In some embodiments, a test device of the disclosure incorporates two or more discrete bodies of porous solid phase material, e.g. separate strips or sheets, each carrying mobile and immobilized reagents. These discrete bodies can be arranged in parallel, for example, such that a single application of liquid sample to the test device initiates sample flow in the discrete bodies simultaneously. The separate analytical results that can be determined in this way can be used as control results. If different reagents are used on the different carriers, the simultaneous determination of a plurality of analytes in a single sample can be made. Alternatively, multiple samples can be applied individually to an array of carriers and analyzed simultaneously.

In other embodiments, a test device is capable of performing two or more lateral flow assays. Each lateral flow assay contained in the device can incorporate one or more solid phase materials, e.g., strips or sheets, each carrying mobile and immobilized reagents.

In some embodiments, the material comprising the porous solid phase is nitrocellulose. This has the advantage that the antibody in the second zone can be immobilized firmly without prior chemical treatment. If the porous solid phase material comprises paper, for example, the immobilization of the antibody in the second zone needs to be performed by chemical coupling using, for example, CNBr, carbonyldiimidazole, or tresyl chloride.

Following the application of the antibody to the detection zone, in some embodiments, the remainder of the porous solid phase material can be treated to block any remaining binding sites elsewhere. Blocking can be achieved by treatment with protein (e.g., bovine serum albumin or milk protein) or with polyvinylalcohol or ethanolamine, or any combination of these agents, for example. The labelled reagent for the first zone can then be dispensed onto the dry carrier and will become mobile in the carrier when in the moist state. Between each of these various process steps (sensitization, application of unlabeled reagent, blocking and application of the labelled reagent), the porous solid phase material is dried.

In some embodiments, the labelled reagent is applied to the carrier as a surface layer rather than being impregnated in the thickness of the carrier, e.g., to assist the free mobility of the labelled reagent when the porous carrier is moistened with the sample. This can minimize interaction between the carrier material and the labelled reagent. In some embodiments, the carrier is pre-treated with a glazing material in the region to which the labelled reagent is to be applied. Glazing can be achieved, for example, by depositing an aqueous sugar or cellulose solution, e.g., of sucrose or lactose, on the carrier at the relevant portion, and drying. The labelled reagent can then be applied to the glazed portion. In some embodiments, the remainder of the carrier material is not be glazed.

In some embodiments, the porous solid phase material is nitrocellulose sheet having a pore size of at least about 1 micron, e.g., greater than about 5 microns (e.g., about 8 to about 12 microns). In other embodiments, the nitrocellulose sheet has a nominal pore size of up to about 12 microns.

In some embodiments, the nitrocellulose sheet is “backed”, e.g., with a plastic sheet, to increase its handling strength. This can be manufactured easily by forming a thin layer of nitrocellulose on a sheet of backing material. The actual pore size of the nitrocellulose when backed in this manner will tend to be, lower than that of the corresponding unbacked material. In some embodiments, a pre-formed sheet of nitrocellulose can be tightly sandwiched between two supporting sheets of solid material, e.g., plastic sheets.

In some embodiments, the flow rate of an aqueous sample through the porous solid phase material is such that in the untreated material, aqueous liquid migrates at a rate of approximately 1 cm in not more than 2 minutes, but slower flow rates can be used if desired. In some embodiments, the spatial separation between the zones, and the flow rate characteristics of the porous carrier material, are selected to allow adequate reaction times during which the necessary specific binding can occur, and to allow the labelled reagent in the first zone to dissolve or disperse in the liquid sample and migrate through the carrier. Further control over these parameters can be achieved by the incorporation of viscosity modifiers (e.g., sugars and modified celluloses) in the sample to slow down the reagent migration.

In yet other embodiments, the immobilized reagent in the second zone is impregnated throughout the thickness of the carrier in the second zone (e.g., throughout the thickness of the sheet or strip if the carrier is in this form). Such impregnation can enhance the extent to which the immobilized reagent can capture any analyte present in the migrating sample.

The reagents can be applied to the carrier material in a variety of ways. Various “printing” techniques can be used to apply liquid reagents to carriers, e.g., micro-syringes, pens using metered pumps, direct printing, and ink-jet printing, and any of these techniques can be used in the present context. To facilitate manufacture, the carrier (e.g., sheet) can be treated with the reagents and then subdivided into smaller portions (e.g., small narrow strips each embodying the required reagent-containing zones) to provide a plurality of identical carrier units.

Accordingly, some embodiments of the disclosure provide a test strip. At one end of the test strip is the sample site to which the sample is to be applied. This sample site comprises a sample pad to which the sample is transferred. Incorporated in the sample site or sample pad, or downstream from the sample site is a labeled specific binding partner (e.g., antibody or antigen), for which the sample is being tested. In some embodiments of the disclosure provided herein, the assay test device comprises a labeled anti-GFAP antibody, labeled anti-UCH-L1 antibody, or labeled anti-GFAP antibody and labeled anti-UCH-L1 antibody.

In some embodiments, the metal sol particles are prepared by coupling the analyte (e.g., GFAP, UCH-L1, or GFAP and UCH-L1) directly to the gold particle. Additionally, the labeled component may be prepared by coupling the analyte to the particle using a biotin/avidin linkage. In this latter regard, the substance may be biotinylated and the metal containing particle coated with an avidin compound. The biotin on the analyte may then be reacted with the avidin compound on the particle to couple the substance and the particle together. In another alternative form of the invention, the labeled component may be prepared by coupling the analyte to a carrier such as bovine serum albumin (BSA), keyhole lymphocyananin (KLH), or ovalbumin and using this to bind to the metal particles.

In some embodiments, the metal sol particles are prepared by methodologies which are well known in the art. For example, the preparation of gold sol particles as disclosed by G. Frens, Nature, 241, 20-22 (1973), the contents of which are herein incorporated by reference, can be used. Additionally, the metal sol particles may comprise metal or metal compounds or polymer nuclei coated with metals or metal compounds, as described in U.S. Pat. No. 4,313,734, the contents of which are herein incorporated herein by reference. Other methods well known in the art may be used to attach the analyte to gold particles. The methods include, but are not limited to, covalent coupling and hydrophobic bonding. The metal sol particles may be made of platinum, gold, silver, selenium, or copper or any number of metal compounds which exhibit characteristic colors.

In some embodiments, the analyte is not attached to a metal sol particle but is instead attached to dyed or fluorescent labeled microparticles such as latex, polystyrene, dextran, silica, polycarbonate, methylmethacrylates, or carbon. The metal sol particles, dyed particles, or fluorescent labeled microparticles should be visible to the naked eye (e.g., as a colored line) or able to be read with an appropriate instrument, or reading device (e.g., a reader) (spectrophotometer, fluorescent reader, etc.). Various embodiments provide a number of ways in which the gold labeled antigens are deposited on the strip. For example, in some embodiments, the gold labeled antigens/antibodies are deposited and dried on a rectangular or square absorbent pad and the absorbent pad is positioned downstream from where the sample is applied on the strip. In other embodiments, the analytes are attached to microspheres. This has the effect of increasing the number of reactive sites (epitopes) in a given area. Analytes may be attached to these alternate solid phases by various methodologies. In some embodiments, hydrophobic or electrostatic domains in the protein are used for passive coating. A suspension of the spheres is mixed after sonication with the antigens/antibodies in water or in a phosphate buffer solution, after which they are incubated at room temperature for 10-75 minutes. The mixture is then centrifuged and the pellets containing the antigen/antibody-linked microspheres are suspended in a buffer containing 1-5% wt/volume bovine serum albumin (BSA) for 1 hour at room temperature. The BSA blocks any unreacted surfaces of the microspheres. After one more centrifugation, the spheres are resuspended in buffer (TBS with 5% BSA) and stored at about 4° C. before using.

In some embodiments, the solid phase particles comprise a known, water dispersible particle, such as, e.g., polystyrene latex particles disclosed in U.S. Pat. No. 3,088,875, incorporated herein by reference. Such solid phase materials simply consist of suspensions of small, water-insoluble particles to which antigens/antibodies are able to bind. Suitable solid phase particles are also disclosed, for example, in U.S. Pat. Nos. 4,184,849, 4,486,530, and 4,636,479, each of which is incorporated herein by reference.

In some embodiments, analytes (e.g., GFAP and/or UCH-L1) are attached to fluorescent microspheres or fluorescent microparticles. Characteristically, fluorescent microspheres incorporate fluorescent dyes in the solid outer matrix or in the internal volume of the microsphere. The fluorescent spheres are typically detected by a fluorescent reading device, or reader that excites molecules at one wavelength and detects the emission of fluorescent waves at another wavelength. For example, Nile Red particles excite at 526 nm at emit at 574 nm, the Far Red excites at 680 nm and emits at 720 nm, and the Blue excites at 365 nm and emits at 430 nm. In a lateral flow format, detection of fluorescent microparticles involves the use of a reflectance reading device or reader with an appropriate excitation source (e.g., HeNe, Argon, tungsten, or diode laser) and an appropriate emission filter for detection. Use of diode lasers allows for use of detection systems that use low-cost lasers with detection above 600 nm. Most background fluorescence is from molecules that emit fluorescence below 550 nm.

In some embodiments, fluorescent microspheres comprise surface functional groups such as carboxylate, sulfate, or aldehyde groups, making them suitable for covalent coupling of proteins and other amine containing biomolecules. In addition, sulfate, carboxyl and amidine microspheres are hydrophobic particles that will passively absorb almost any protein or lectin. Coating is thus similar as for nonfluorescent microspheres. In some embodiments, a suspension of the fluorescent spheres is mixed after sonication with the antigens/antibody in water or in a phosphate buffered solution, after which they are incubated at room temperature for about 10 to about 75 minutes. EDAC (soluble carbodiimide), succinimidyl esters and isothiocyanates as well as other crosslinking agents may be used for covalent coupling of proteins and lectins to the microspheres. After the protein has attached to the surface of the microparticles, the mixture is centrifuged and the pellets containing the antigen or antibody linked to the fluorescent microparticles are suspended in a buffer containing 1-5% bovine serum albumin for one hour. After one more centrifugation, the spheres are resuspended in buffer (TBS with 5% BSA or other appropriate buffers) and stored at about 4° C. before use.

In some embodiments, the solid phase particles comprise, for example, particles of latex or of other support materials such as silica, agarose, glass, polyacrylamides, polymethyl methacrylates, carboxylate modified latex and Sepharose. Preferably, the particles vary in size from about 0.2 microns to about 10 microns. In some embodiments, particles are coated with a layer of antigens coupled thereto in a manner known per se in the art to present the solid phase component.

Accordingly, other embodiments involve providing a sample suspected of containing GFAP, UCH-L1, or GFAP and UCH-L1 that reacts with a first labeled antibody (e.g., an anti-GFAP antibody, anti-UCH-L1 antibody, or anti-GFAP antibody and anti-UCH-L1 antibody) on the test strip to form a first antibody-GFAP, UCH-L1 or GFAP and UCH-L1 complex. After formation, the first antibody-GFAP, UCH-L1 or GFAP and UCH-L1 complex begins to progress along the test strip into or through a detection zone in the test strip which contains at least one second antibody that binds to a different epitope than the first antibody and forms a first labeled antibody-GFAP, UCH-L1, or GFAP and UCH-L1-second antibody complex which is detected.

In yet other embodiments, the test strip comprises three binding sites. For example, the first binding site binds GFAP or UCH-L1. A second binding site binds UCH-L1 or GFAP, whichever is not bound at the first binding site. A third binding site is for a control. More specifically, each binding site is in the form of a striped line along the width of the test strip. Each binding site comprises an antibody. By way of another example, in some embodiments, an anti-GFAP antibody or anti-UCH-L1 antibody is laid down at the first binding site and an anti-UCH-L1 or anti-GFAP antibody is laid down at the second site. One or both antibodies at the first and second binding sites can be labeled with a detectable label. At the control site, there is immobilized an antibody to a control substance (e.g., a labeled antibody or antigen).

Thus provided herein are at least one lateral flow assay for GFAP and/or at least one lateral flow assay for UCH-L1. In some embodiments, the at least one lateral flow assay for GFAP and/or the at least one lateral flow assay for UCH-L1 are each performed or capable of being performed in less than about 30 minutes. In yet some other embodiments, the at least one lateral flow assay for GFAP and/or the at least one lateral flow assay for UCH-L1 are each are each performed or capable of being performed in less than about 25 minutes. In still further embodiments, the at least one lateral flow assay for GFAP and/or the at least one lateral flow assay for UCH-L1 are each are each performed or capable of being performed in about less than about 20 minutes. In still yet other embodiments, the at least one lateral flow assay for GFAP and/or the at least one lateral flow assay for UCH-L1 are each are each performed or capable of being performed in less than about 18 minutes. In still yet other embodiments, the at least one lateral flow assay for GFAP and/or the at least one lateral flow assay for UCH-L1 are each are each performed or capable of being performed in less than about 15 minutes. In still yet other embodiments, the at least one lateral flow assay for GFAP and/or the at least one lateral flow assay for UCH-L1 are each are each performed or capable of being performed in a time ranging from about 4 to about 20 minutes, optionally in a time ranging from about 10 to about 15 minutes. In still further embodiments, the at least one lateral flow assay for GFAP and/or the at least one lateral flow assay for UCH-L1 are each are each performed or capable of being performed in a time ranging about 15 to about 18 minutes.

In some embodiments, the at least one lateral flow assay for GFAP and/or at the at least one lateral flow assay for UCH-L1 are each performed or capable of being performed in about 4 minutes. In some embodiments, the at least one lateral flow assay for GFAP and at the at least one lateral flow assay for UCH-L1 are each performed or capable of being performed in about 5 minutes. In some embodiments, the at least one lateral flow assay for GFAP and/or at the at least one lateral flow assay for UCH-L1 are each performed or capable of being performed in about 6 minutes. In some embodiments, the at least one lateral flow assay for GFAP and/or at the at least one lateral flow assay for UCH-L1 are each performed or capable of being performed in about 7 minutes. In some embodiments, the at least one lateral flow assay for GFAP and/or at the at least one lateral flow assay for UCH-L1 are each performed or capable of being performed in about 8 minutes. In some embodiments, the at least one lateral flow assay for GFAP and/or at the at least one lateral flow assay for UCH-L1 are each performed or capable of being performed in about 9 minutes. In some embodiments, the at least one lateral flow assay for GFAP and/or at the at least one lateral flow assay for UCH-L1 are each performed or capable of being performed in about 10 minutes. In some embodiments, the at least one lateral flow assay for GFAP and/or at the at least one lateral flow assay for UCH-L1 are each performed or capable of being performed in about 11 minutes. In some embodiments, the at least one lateral flow assay for GFAP and/or at the at least one lateral flow assay for UCH-L1 are each performed or capable of being performed in about 12 minutes. In some embodiments, the at least one lateral flow assay for GFAP and/or at the at least one lateral flow assay for UCH-L1 are each performed or capable of being performed in about 13 minutes. In some embodiments, the at least one lateral flow assay for GFAP and/or at the at least one lateral flow assay for UCH-L1 are each performed or capable of being performed in about 14 minutes. In some embodiments, the at least one lateral flow assay for GFAP and/or at the at least one lateral flow assay for UCH-L1 are each performed or capable of being performed in about 15 minutes. In some embodiments, the at least one lateral flow assay for GFAP and/or at the at least one lateral flow assay for UCH-L1 are each performed or capable of being performed in about 16 minutes. In some embodiments, the at least one lateral flow assay for GFAP and/or at the at least one lateral flow assay for UCH-L1 are each performed or capable of being performed in about 17 minutes. In some embodiments, the at least one lateral flow assay for GFAP and/or at the at least one lateral flow assay for UCH-L1 are each performed or capable of being performed in about 18 minutes. In some embodiments, the at least one lateral flow assay for GFAP and/or at the at least one lateral flow assay for UCH-L1 are each performed or capable of being performed in about 19 minutes. In some embodiments, the at least one lateral flow assay for GFAP and/or at the at least one lateral flow assay for UCH-L1 are each performed or capable of being performed in about 20 minutes. In some embodiments, the at least one lateral flow assay for GFAP and/or at the at least one lateral flow assay for UCH-L1 are each performed or capable of being performed in about 25 minutes. In some embodiments, the at least one lateral flow assay for GFAP and/or at the at least one lateral flow assay for UCH-L1 are each performed or capable of being performed in about 30 minutes.

In another embodiment, the present disclosure relates to a system comprising a lateral flow device or test strip, a reading device or reader, a data analyzer. and a memory. The reading device or reader comprises a port or opening for receiving the lateral flow device or a test strip from a lateral flow device. When the lateral flow device or a test strip from the lateral flow device is loaded into the port or opening, the reading device or reader obtains light intensity measurements from the device or test strip. In some embodiments, the light intensity measurements may be unfiltered or filtered with respect to at least one wavelength and polarization. The data analyzer computes at least one parameter from one or more of the light intensity measurements. The result of an assay performed on the test strip can be communicated by the reading device or reader. In other embodiments, the system described herein does not contain a reading device or reader. In such embodiments, the system my comprise a lateral flow device or test strip and a computer with memory. The results of an assay performed on the test strip can be inputted into the computer.

In other embodiments, the disclosure relates to methods of using the lateral flow assays and lateral flow devices described herein for determining the presence or amount of GFAP, UCH-L1 or GFAP and UCH-L1 in a sample obtained from a subject to assess, determine and/or diagnose whether a subject has suffered an injury and/or is suffering from a disease or other medical condition. For example, determining the presence or amount of GFAP, UCH-L1 or GFAP and UCH-L1 can be used to assess and/or determine whether a subject has sustained an injury to the head (e.g., such as a traumatic brain injury), has suffered a stroke (such as an ischemic stroke), has SARS-CoV-2, or has Alzheimer's disease. By way of another example, determining the presence or amount of GFAP can be used to assess, determine and/or diagnose whether a subject has sustained an injury to the head (e.g., such as a traumatic brain injury), has suffered a stroke (such as an ischemic stroke), an intracerebral hemorrhage, or astrocytic injury (such as that caused by SARS-CoV-2), or has Alzheimer's disease, Alexander disease, cancer (e.g., such as glioblastoma), or infection (such as Toxocara ova, lyme neuroborreliosis, etc.). By way of still another example, determining the presence or amount of UCH-L1 can be used to assess, determine and/or diagnose whether a subject has sustained an injury to the head (e.g., such as a traumatic brain injury), has suffered a stroke (such as an ischemic stroke), neuronal apotosis (e.g, such as that induced by deep hypothermic circulatory arrest), or has white matter lesions (subcortical), Parkinson's disease, or Alzheimer's disease. In some embodiments, the methods are performed using an immunoassay. The immunoassay may be an enzyme-linked immunosorbent assay (ELISA) or a lateral flow immunoassay (LFA).

In still yet other embodiments, this disclosure relates to methods for determining the presence or amount of GFAP, UCH-L1, or GFAP and UCH-L1 in a sample obtained from a subject who may have sustained or has sustained an injury to the head using the lateral flow assays and lateral flow devices described herein. In some embodiments, the lateral flow assay is an immunoassay. In some embodiments, the methods involve detecting the presence of GFAP, UCH-L1, or GFAP and UCH-L1 in a sample obtained from a subject by performing a lateral flow assay. In some embodiments, the lateral flow assay is an immunoassay. In other embodiments, the methods involve determining whether the level of GFAP, UCH-L1, or GFAP and UCH-L1 in a sample obtained from a subject is elevated by performing a lateral flow assay. In some embodiments, the lateral flow assay is an immunoassay. In yet other embodiments, the determination of: (1) the presence of GFAP, UCH-L1, or GFAP and UCH-L1 in a sample; or (2) whether a subject's levels of GFAP, UCH-L1, or GFAP and UCH-L1 are elevated, aids in the diagnosis and evaluation of whether the subject has sustained an injury to the head. In some embodiments, the methods for determining: (1) the presence of GFAP, UCH-L1, or GFAP and UCH-L1 in a sample; or (2) whether a subject's levels of GFAP, UCH-L1, or GFAP and UCH-L1 are elevated, can aid in the determination of whether or not a subject requires further evaluation, such as by a head computed tomography (CT) scan and/or a magnetic resonance imaging (MRI) procedure.

In some embodiments, the visual limit of detection (LOD) for GFAP is between about 0.5 pg/mL to about 1250 pg/mL, about 1 pg/mL to about 1250 pg/mL, about 2 pg/mL to about 1250 pg/mL, about 3 pg/mL to about 1250 pg/mL, about 4 pg/mL to about 1250 pg/mL, about 5 pg/mL to about 1250 pg/mL, about 6 pg/mL to about 1250 pg/mL, about 7 pg/mL to about 1250 pg/mL, about 8 pg/mL to about 1250 pg/mL, about 9 pg/mL to about 1250 pg/mL, about 10 pg/mL to about 1250 pg/mL, or about 15 pg/mL to about 1250 pg/mL and/or the visual limit of detection for UCH-L1 is between about 0.5 pg/mL to about 1250 pg/mL, about 1 pg/mL to about 1250 pg/mL, about 2 pg/mL to about 1250 pg/mL, about 3 pg/mL to about 1250 pg/mL, about 4 pg/mL to about 1250 pg/mL, about 5 pg/mL to about 1250 pg/mL, about 6 pg/mL to about 1250 pg/mL, about 7 pg/mL to about 1250 pg/mL, about 8 pg/mL to about 1250 pg/mL, about 9 pg/mL to about 1250 pg/mL, about 10 pg/mL to about 1250 pg/mL about 15 pg/mL to about 1250 pg/mL, about 20 pg/mL to about 1250 pg/mL, about 25 pg/mL to about 1250 pg/mL, about 50 pg/mL to about 1250 pg/mL, about 100 pg/mL to about 1250 pg/mL or 200 pg/mL to about 1250 pg/mL. In some other embodiments, the visual limit of detection for GFAP is between about 20 pg/mL to about 125 pg/mL and/or the visual limit of detection for UCH-L1 is between about 250 pg/mL to about 1250 pg/mL In some other embodiments, the visual limit of detection for GFAP is between about 25 pg/mL to about 125 pg/mL and/or the visual limit of detection for UCH-L1 is between about 300 pg/mL to about 1250 pg/mL In yet other embodiments, the visual limit of detection for GFAP is between about 30 pg/mL to about 125 pg/mL and/or the visual limit of detection for UCH-L1 is between about 300 pg/mL to about 1250 pg/mL In yet still other embodiments, the visual limit of detection for GFAP is between about 35 pg/mL to about 125 pg/mL and/or the visual limit of detection for UCH-L1 is between about 350 pg/mL to about 1250 pg/mL. In yet further embodiments, the visual limit of detection for GFAP is between about 35 pg/mL to about 100 pg/mL and/or the visual limit of detection for UCH-L1 is between about 350 pg/mL to about 750 pg/mL. In still yet other embodiments, the visual limit of detection for GFAP is between about 35 pg/mL to about 50 pg/mL and/or the visual limit of detection for UCH-L1 is between about 35 pg/mL to about 500 pg/mL. In yet further embodiments, with optimization (e.g., of the assay, assay components, visual display, or conversion to a numerical value), the visual limit of detection for GFAP, UCH-L1 and GFAP and UCH-L1, can be reduced further, such as, for example, to be 5-fold more sensitive, 10-fold more sensitive, 20-fold more sensitive, 25-fold more sensitive, 30-fold more sensitive, 40-fold more sensitive, 50-fold more sensitive, 60-fold more sensitive, 70-fold more sensitive, 80-fold more sensitive, 90-fold more sensitive, or 100-fold more sensitive.

The methods described herein utilize at least one sample obtained from a subject (e.g., from the human subject). In some embodiments, the sample is obtained within about 48 hours after an actual or suspected injury to the head. In other embodiments, the sample is obtained within about 24 hours after an actual or suspected injury to the head. In yet other embodiments, the sample is obtained within about 12 hours after an actual or suspected injury to the head. In some embodiments, the sample is taken from the subject (e.g., human subject) within about 48 hours of injury of an actual or suspected injury to the head. For example, the sample can be taken from the subject (e.g., a human subject) within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, or within about 48 hours after an actual or suspected injury to the head.

In other embodiments, the methods, assays, and lateral flow devices described herein further comprise performing a head computed tomography (CT) scan, a magnetic resonance imaging (MRI) procedure, or both a CT scan or a MRI procedure on the subject when the presence of GFAP, UCH-L1 or GFAP and UCH-L1 is detected in a sample from a subject. For example, in yet further embodiments, the methods further comprise performing a head CT scan on the subject when the presence of GFAP, UCH-L1 or GFAP and UCH-L1 is detected in a sample from the subject. In still further embodiments, the methods further comprise performing a MRI procedure on the subject when the presence of GFAP, UCH-L1 or GFAP and UCH-L1 is detected in a sample from the subject. In yet further embodiments in yet further embodiments, the methods further comprise performing a head CT scan and a MRI procedure on the subject when the presence of GFAP, UCH-L1 or GFAP and UCH-L1 is detected in a sample from the subject.

In still other embodiments, the methods described herein further comprises performing a head computed tomography (CT) scan, a magnetic resonance imaging (MRI) procedure, or both a CT scan or a MRI procedure on the subject when the subject's levels of GFAP, UCH-L1, or GFAP and UCH-L1 are elevated. For example, in some embodiments, the methods further comprise performing a head CT scan on the subject when the subject's levels of GFAP, UCH-L1, or GFAP and UCH-L1 are elevated. As another example, in some embodiments, the methods further comprise performing an MRI procedure on the subject when the subject's levels of GFAP, UCH-L1, or GFAP and UCH-L1 are elevated. In yet other embodiments, the method further comprises performing a head CT scan and an MRI procedure on the subject when the subject's levels of GFAP, UCH-L1, or GFAP and UCH-L1 are elevated.

In other embodiments, methods further comprise not performing a head computed tomography (CT) scan, a magnetic resonance imaging (MRI) procedure, or both a head CT scan or a MRI procedure on the subject when the presence of GFAP, UCH-L1 or GFAP and UCH-L1 in the sample is not detected. In still yet other embodiments, the methods further comprise not performing a head computed tomography (CT) scan, a magnetic resonance imaging (MRI) procedure, or both a head CT scan or a MRI procedure on the subject when the subject's levels of GFAP, UCH-L1, or GFAP and UCH-L1 are not elevated. In other words, the methods involve “ruling out” the need for a head CT scan, a MRI procedure or both when (1) the presence of GFAP, UCH-L1, or GFAP and UCH-L1 is not detected in a sample; or (2) the subject's GFAP, UCH-L1, or GFAP and UCH-L1 levels are not elevated.

In some embodiments, the methods further comprise treating the subject for a mild, moderate, moderate to severe, or severe TBI when (1) the presence of GFAP, UCH-L1, or GFAP and UCH-L1 is detected in a sample of the subject; or (2) the subject's levels of GFAP, UCH-L1, or GFAP and UCH-L1 are determined to be elevated. For example, in some embodiments, the methods further comprise treating the subject for a mild TBI when (1) the presence of GFAP, UCH-L1, or GFAP and UCH-L1 is detected in a sample of the subject; or (2) the subject's levels of GFAP, UCH-L1, or GFAP and UCH-L1 are determined to be elevated. In some embodiments, the method further comprises treating the subject for a moderate to severe TBI when (1) the presence of GFAP, UCH-L1, or GFAP and UCH-L1 is detected in the sample of the subject; or (2) the subject's levels of GFAP, UCH-L1, or GFAP and UCH-L1 are determined to be elevated. In some embodiments, the method further comprises treating the subject for a severe TBI when (1) the presence of GFAP, UCH-L1, or GFAP and UCH-L1 is detected in the sample of the subject; or (2) the subject's levels of GFAP, UCH-L1, or GFAP and UCH-L1 are determined to be elevated. In some embodiments, selection of the appropriate treatment may be facilitated by results from a head CT scan, an MRI procedure, or both, if performed on the subject. For example, results from a head CT scan and/or MRI procedure may help in further differentiating between a mild, moderate to severe, or a severe TBI in the subject. Such a differentiation may assist in selection of the appropriate treatment for the subject. In some embodiments, the method further comprises monitoring the subject when the subject's levels of GFAP, UCH-L1, or GFAP and UCH-L1 are elevated.

In some embodiments, the method further includes treating a subject (e.g., a human subject) assessed as having mild, moderate, severe, or moderate to severe traumatic brain injury with a traumatic brain injury treatment, as described below. In yet other embodiments, the method further includes treating a subject (e.g., a human subject) assessed with a mild traumatic brain injury with traumatic brain injury treatment, as described below. In yet other embodiments, the method further includes treating a subject (e.g., a human subject) assessed with moderate traumatic brain injury with traumatic brain injury treatment, as described below. In yet other embodiments, the method further includes treating a subject assessed with severe traumatic brain injury with a traumatic brain injury treatment. In some embodiments, the method further includes monitoring a subject (e.g., a human subject) assessed as having mild traumatic brain injury, as described below. In other embodiments, the method further includes monitoring a subject (e.g., a human subject) assessed as having a moderate traumatic brain injury, as described below. In yet other embodiments, the method further includes monitoring a subject (e.g., a human subject) assessed as having a severe traumatic brain injury, as described below. In yet other embodiments, the method further includes monitoring a subject (e.g., a human subject) assessed as having a moderate to severe traumatic brain injury.

### 3. Treatment and Monitoring of a Subject Suffering from Traumatic Brain Injury

The subject (e.g., a human subject) identified or assessed in the methods, lateral flow assays, and lateral flow devices described herein may be treated or monitored when: (1) the presence of GFAP, UCH-L1, or GFAP and UCH-L1 is detected; or (2) the levels of GFAP, UCH-L1, or GFAP and UCH-L1 are determined to be elevated. In some embodiments, the methods further include treating the subject (e.g., human subject) with a traumatic brain injury treatment, such as any treatments known in the art, where: (1) the presence of GFAP, UCH-L1, or GFAP and UCH-L1 in a sample is detected; or (2) the subject is determined as having elevated levels of GFAP, UCH-L1, or GFAP and UCH-L1. For example, treatment of traumatic brain injury can take a variety of forms depending on the severity of the injury to the head. For example, for subjects suffering from mild TBI, the treatment may include one or more of rest, abstaining from physical activities, such as sports, avoiding light or wearing sunglasses when out in the light, medication for relief of a headache or migraine, anti-nausea medication, etc. Treatment for patients suffering from moderate, severe, or moderate to severe TBI might include administration of one or more appropriate medications (such as, for example, diuretics, anti-convulsant medications, medications to sedate and put an individual in a drug-induced coma, or other pharmaceutical or biopharmaceutical medications (either known or developed in the future for treatment of TBI), one or more surgical procedures (such as, for example, removal of a hematoma, repairing a skull fracture, decompressive craniectomy, etc.), protecting the airway, and one or more therapies (such as, for example one or more rehabilitation, cognitive behavioral therapy, anger management, counseling psychology, etc.). In some embodiments, the method further includes monitoring the subject (e.g., a human subject): (1) where the presence of GFAP, UCH-L1, or GFAP and UCH-L1 is detected in a sample (e.g., which may be indicative or mild, moderate, severe, or moderate to severe traumatic brain injury, or mild, moderate, severe, or moderate to severe traumatic brain injury); or (2) assessed having elevated levels of GFAP, UCH-L1, or GFAP and UCH-L1 (e.g., which may be indicative or mild, moderate, severe, or moderate to severe traumatic brain injury, or mild, moderate, severe, or moderate to severe traumatic brain injury). For example, monitoring the subject: (1) where the presence of GFAP, UCH-L1, or GFAP and UCH-L1 is detected in a sample; or (2) assessed as having elevated levels of GFAP, UCH-L1, or GFAP and UCH-L1 may comprise monitoring with a CT scan and/or a MRI procedure. In some embodiments, a subject identified as having traumatic brain injury, such as mild traumatic brain injury, moderate traumatic brain injury, severe traumatic brain injury, or moderate to severe traumatic brain injury or mild traumatic brain injury, moderate traumatic brain injury, severe traumatic brain injury, or moderate to severe traumatic brain injury may be monitored with CT scan and/or MRI.

### 4. Methods for Measuring the Level of UCH-L1

In the methods, lateral flow assays, and lateral flow devices described above, UCH-L1 levels can be measured by any means. In some embodiments, measuring the presence or amount of UCH-L1 includes contacting the sample with a first specific binding member and second specific binding member. In some embodiments, the first specific binding member is a capture antibody and the second specific binding member is a detection antibody. In some embodiments, measuring the level of UCH-L1 includes contacting the sample, either simultaneously or sequentially, in any order: (1) a capture antibody (e.g., UCH-L1-capture antibody), which binds to an epitope on UCH-L1 or UCH-L1 fragment to form a capture antibody-UCH-L1 antigen complex (e.g., UCH-L1-capture antibody-UCH-L1 antigen complex), and (2) a detection antibody (e.g., UCH-L1-detection antibody), which includes a detectable label and binds to an epitope on UCH-L1 that is not bound by the capture antibody, to form a UCH-L1 antigen-detection antibody complex (e.g., UCH-L1 antigen-UCH-L1-detection antibody complex), such that a capture antibody-UCH-L1 antigen-detection antibody complex (e.g., UCH-L1-capture antibody-UCH-L1 antigen-UCH-L1-detection antibody complex) is formed, and measuring the amount or concentration of UCH-L1 in the sample based on the signal generated by the detectable label in the capture antibody-UCH-L1 antigen-detection antibody complex.

In some embodiments, the first specific binding member is immobilized on a solid support. In some embodiments, the second specific binding member is immobilized on a solid support. In some embodiments, the first specific binding member is a UCH-L1 antibody as described below.

In some embodiments, the sample is diluted or undiluted. In some embodiments, the sample is about 1 to about 100 microliters. In some embodiments, the sample is about 10 to about 90 microliters. In some embodiments, the sample is about 1 microliter, about 5 microliters, about 10 microliters, about 20 microliters, about 30 microliters, about 40 microliters, about 50 microliters, about 60 microliters, about 70 microliters, about 80 microliters, or about 90 microliters In some embodiments, the sample is from about 1 to about 85 microliters, about 1 to about 80 microliters, about 1 to about 75 microliters, about 1 to about 65 microliters, about 1 to about 50 microliters, about 1 to about 40 microliters, about 1 to about 30 microliters, about 1 to about 20 microliters, about 1 to about 10 microliters, or about 1 to about 5 microliters. In some embodiments, the sample is about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters, about 25 microliters, about 26 microliters, about 27 microliters, about 28 microliters, about 29 microliters, about 30 microliters, about 40 microliters, about 50 microliters, about 60 microliters, about 70 microliters, about 80 microliters, about 90 microliters, or about 100 microliters. In some embodiments, the sample is from about 1 to about 150 microliters or less or from about 1 to about 80 microliters or less.

### 5. UCH-L1 Antibodies

The methods described herein may use an isolated antibody that specifically binds to ubiquitin carboxy-terminal hydrolase L1 (“UCH-L1”) (or fragments thereof), referred to as “UCH-L1 antibody.” The UCH-L1 antibodies can be used to assess the UCH-L1 status as a measure of traumatic brain injury, detect the presence of UCH-L1 in a sample, quantify the amount of UCH-L1 present in a sample, or detect the presence of and quantify the amount of UCH-L1 in a sample.

a. Ubiquitin Carboxy-Terminal Hydrolase L1 (UCH-L1)

Ubiquitin carboxy-terminal hydrolase L1 (“UCH-L1”), which is also known as “ubiquitin C-terminal hydrolase,” is a deubiquitinating enzyme. UCH-L1 is a member of a gene family whose products hydrolyze small C-terminal adducts of ubiquitin to generate the ubiquitin monomer. Expression of UCH-L1 is highly specific to neurons and to cells of the diffuse neuroendocrine system and their tumors. It is abundantly present in all neurons (accounts for 1-2% of total brain protein), expressed specifically in neurons and testis/ovary. The catalytic triad of UCH-L1 contains a cysteine at position 90, an aspartate at position 176, and a histidine at position 161 that are responsible for its hydrolase activity.

Human UCH-L1 may have the following amino acid sequence:

(SEQ ID NO: 1)
MQLKPMEINPEMLNKVLSRLGVAGQWRFVDVLGLEEESLGSVPAPACAL
LLLFPLTAQHENFRKKQIEELKGQEVSPKVYFMKQTIGNSCGTIGLIHA
VANNQDKLGFEDGSVLKQFLSETEKMSPEDRAKCFEKNEAIQAAHDAVA
QEGQCRVDDKVNFHFILFNNVDGHLYELDGRMPFPVNHGASSEDTLLKD
AAKVCREFTEREQGEVRESAVALCKAA.

The human UCH-L1 may be a fragment or variant of SEQ ID NO: 1. The fragment of UCH-L1 may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise a contiguous number of amino acids from SEQ ID NO: 1.

b. UCH-L1-Recognizing Antibody

The antibody is an antibody that binds to UCH-L1, a fragment thereof, an epitope of UCH-L1, or a variant thereof. The antibody may be a fragment of the anti-UCH-L1 antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-UCH-L1 antibodies may be a chimeric anti-UCH-L1 or humanized anti-UCH-L1 antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008(8):85. Therefore, the antibody may be a product of the human and not animal repertoire. Because it is of human origin, the risks of reactivity against self-antigens may be minimized. Alternatively, standard yeast display libraries and display technologies may be used to select and isolate human anti-UCH-L1 antibodies. For example, libraries of naïve human single chain variable fragments (scFv) may be used to select human anti-UCH-L1 antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to UCH-L1 (SEQ ID NO: 1), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77: 4216-4220 (1980)), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human UCH-L1) and the other heavy and light chain are specific for an antigen other than human UCH-L1 by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with UCH-L1 or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass); Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); Hawkins et al, J. Mol. Biol., 226: 889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (1311), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

### (1) Anti-UCH-L1 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with UCH-L1 with myeloma cells and then screening the hybridomas resulting from the fusion for hybridoma clones that secrete an antibody able to bind a polypeptide of the disclosure. Briefly, rats can be immunized with a UCH-L1 antigen. In a preferred embodiment, the UCH-L1 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a UCH-L1 antigen, antibodies and/or antibody-producing cells may be obtained from the animal. An anti-UCH-L1 antibody-containing serum is obtained from the animal by bleeding or sacrificing the animal. The serum may be used as it is obtained from the animal, an immunoglobulin fraction may be obtained from the serum, or the anti-UCH-L1 antibodies may be purified from the serum. Serum or immunoglobulins obtained in this manner are polyclonal, thus having a heterogeneous array of properties.

Once an immune response is detected, e.g., antibodies specific for the antigen UCH-L1 are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding UCH-L1. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using UCH-L1, or a portion thereof, or a cell expressing UCH-L1. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-UCH-L1 antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-UCH-L1 antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

### (2) Anti-UCH-L1 Monoclonal Antibodies Using SLAM

In another embodiment of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93: 7843-7848 (1996). In this method, single cells secreting antibodies of interest, e.g., lymphocytes derived from any one of the immunized animals are screened using an antigen-specific hemolytic plaque assay, wherein the antigen UCH-L1, a subunit of UCH-L1, or a fragment thereof, is coupled to sheep red blood cells using a linker, such as biotin, and used to identify single cells that secrete antibodies with specificity for UCH-L1. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to UCH-L1. The amplified immunoglobulin sequences further can be manipulated in vitro, such as by in vitro affinity maturation method. See, for example, PCT Publication No. WO 97/29131 and PCT Publication No. WO 00/56772.

### (3) Anti-UCH-L1 Monoclonal Antibodies Using Transgenic Animals

In another embodiment of the disclosure, antibodies are produced by immunizing a non-human animal comprising some, or all, of the human immunoglobulin locus with a UCH-L1 antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7: 13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15: 146-156 (1997), Green and Jakobovits, J. Exp. Med., 188: 483-495 (1998), the disclosures of which are hereby incorporated by reference.

### (4) Anti-UCH-L1 Monoclonal Antibodies Using Recombinant Antibody Libraries

In vitro methods also can be used to make the antibodies of the disclosure, wherein an antibody library is screened to identify an antibody having the desired UCH-L1-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9: 1369-1372 (1991); Hay et al., Hum. Antibod. Hybridomas, 3: 81-85 (1992); Huse et al., Science, 246: 1275-1281 (1989); McCafferty et al., Nature, 348: 552-554 (1990); Griffiths et al., EMBO J., 12: 725-734 (1993); Hawkins et al., J. Mol. Biol., 226: 889-896 (1992); Clackson et al., Nature, 352: 624-628 (1991); Gram et al., Proc. Natl. Acad. Sci. USA, 89: 3576-3580 (1992); Garrard et al., Bio/Technology, 9: 1373-1377 (1991); Hoogenboom et al., Nucl. Acids Res., 19: 4133-4137 (1991); Barbas et al., Proc. Natl. Acad. Sci. USA, 88: 7978-7982 (1991); U.S. Patent Application Publication No. 2003/0186374; and PCT Publication No. WO 97/29131, the contents of each of which are incorporated herein by reference.

The recombinant antibody library may be from a subject immunized with UCH-L1, or a portion of UCH-L1. Alternatively, the recombinant antibody library may be from a naive subject, i.e., one who has not been immunized with UCH-L1, such as a human antibody library from a human subject who has not been immunized with human UCH-L1. Antibodies of the disclosure are selected by screening the recombinant antibody library with the peptide comprising human UCH-L1 to thereby select those antibodies that recognize UCH-L1. Methods for conducting such screening and selection are well known in the art, such as described in the references in the preceding paragraph. To select antibodies of the disclosure having particular binding affinities for UCH-L1, such as those that dissociate from human UCH-L1 with a particular K_{off} rate constant, the art-known method of surface plasmon resonance can be used to select antibodies having the desired K_{off} rate constant. To select antibodies of the disclosure having a particular neutralizing activity for hUCH-L1, such as those with a particular IC₅₀, standard methods known in the art for assessing the inhibition of UCH-L1 activity may be used.

In one embodiment, the disclosure pertains to an isolated antibody, or an antigen-binding portion thereof, that binds human UCH-L1. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182: 41-50 (1995); Ames et al., J. Immunol. Methods, 184:177-186 (1995); Kettleborough et al., Eur. J. Immunol., 24: 952-958 (1994); Persic et al., Gene, 187: 9-18 (1997); Burton et al., Advances in Immunology, 57: 191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12(6): 864-869 (1992); Sawai et al., Am. J. Reprod. Immunol., 34: 26-34 (1995); and Better et al., Science, 240: 1041-1043 (1988). Examples of techniques which can be used to produce single-chain Fvs and antibodies include those described in U.S. Pat. Nos. 4,946,778 and 5,258,498; Huston et al., Methods in Enzymology, 203: 46-88 (1991); Shu et al., Proc. Natl. Acad. Sci. USA, 90: 7995-7999 (1993); and Skerra et al., Science, 240: 1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94: 12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant UCH-L1 Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77: 4216-4220 (1980), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human UCH-L1) and the other heavy and light chain are specific for an antigen other than human UCH-L1 by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for UCH-L1 and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for UCH-L1, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-UCH-L1 Antibodies

Anti-UCH-L1 antibodies may be generated using the techniques described above as well as using routine techniques known in the art. In some embodiments, the anti-UCH-L1 antibody may be an unconjugated UCH-L1 antibody, such as UCH-L1 antibodies available from United State Biological (Catalog Number: 031320), Cell Signaling Technology (Catalog Number: 3524), Sigma-Aldrich (Catalog Number: HPA005993), Santa Cruz Biotechnology, Inc. (Catalog Numbers: sc-58593 or sc-58594), R&D Systems (Catalog Number: MAB6007), Novus Biologicals (Catalog Number: NB600-1160), Biorbyt (Catalog Number: orb33715), Enzo Life Sciences, Inc. (Catalog Number: ADI-905-520-1), Bio-Rad (Catalog Number: VMA00004), BioVision (Catalog Number: 6130-50), Abcam (Catalog Numbers: ab75275 or ab104938), Invitrogen Antibodies (Catalog Numbers: 480012), ThermoFisher Scientific (Catalog Numbers: MA1-46079, MA5-17235, MA1-90008, or MA1-83428), EMD Millipore (Catalog Number: MABN48), or Sino Biological Inc. (Catalog Number: 50690-RO11). The anti-UCH-L1 antibody may be conjugated to a fluorophore, such as conjugated UCH-L1 antibodies available from BioVision (Catalog Number: 6960-25) or Aviva Systems Biology (Cat. Nos. OAAF01904-FITC).

Alternatively, the antibodies described in WO 2018/067474, WO2018/081649, U.S. Pat. No. 11,078,298, U.S. Patent Publication No. 2019/0502127, and/or Bazarian et al., “Accuracy of a rapid GFAP/UCH-L1 test for the prediction of intracranial injuries on head CT after mild traumatic brain injury”, Acad. Emerg. Med., (Aug. 6, 2021), the contents of which are herein incorporated by reference, can also be used.

### 6. Methods for Measuring the Level of GFAP

In the methods described above, GFAP levels can be measured by any means. In some embodiments, measuring the level of GFAP includes contacting the sample with a first specific binding member and second specific binding member. In some embodiments, the first specific binding member is a capture antibody and the second specific binding member is a detection antibody. In some embodiments, measuring the level of GFAP includes contacting the sample, either simultaneously or sequentially, in any order: (1) a capture antibody (e.g., GFAP-capture antibody), which binds to an epitope on GFAP or GFAP fragment to form a capture antibody-GFAP antigen complex (e.g., GFAP-capture antibody-GFAP antigen complex), and (2) a detection antibody (e.g., GFAP-detection antibody), which includes a detectable label and binds to an epitope on GFAP that is not bound by the capture antibody, to form a GFAP antigen-detection antibody complex (e.g., GFAP antigen-GFAP-detection antibody complex), such that a capture antibody-GFAP antigen-detection antibody complex (e.g., GFAP-capture antibody-GFAP antigen-GFAP-detection antibody complex) is formed, and measuring the amount or concentration of GFAP in the sample based on the signal generated by the detectable label in the capture antibody-GFAP antigen-detection antibody complex.

In some embodiments, the first specific binding member is immobilized on a solid support. In some embodiments, the second specific binding member is immobilized on a solid support. In some embodiments, the first specific binding member is a GFAP antibody as described below.

In some embodiments, the sample is diluted or undiluted. In some embodiments, the sample is about 1 to about 100 microliters. In some embodiments, the sample is about 10 to about 90 microliters. In some embodiments, the sample is about 1 microliter, about 5 microliters, about 10 microliters, about 20 microliters, about 30 microliters, about 40 microliters, about 50 microliters, about 60 microliters, about 70 microliters, about 80 microliters, or about 90 microliters In some embodiments, the sample is from about 1 to about 85 microliters, about 1 to about 80 microliters, about 1 to about 75 microliters, about 1 to about 65 microliters, about 1 to about 50 microliters, about 1 to about 40 microliters, about 1 to about 30 microliters, about 1 to about 20 microliters, about 1 to about 10 microliters, or about 1 to about 5 microliters. In some embodiments, the sample is about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters, about 25 microliters, about 26 microliters, about 27 microliters, about 28 microliters, about 29 microliters, about 30 microliters, about 40 microliters, about 50 microliters, about 60 microliters, about 70 microliters, about 80 microliters, about 90 microliters, or about 100 microliters. In some embodiments, the sample is from about 1 to about 150 microliters or less or from about 1 to about 80 microliters or less. GFAP Antibodies

The methods described herein may use an isolated antibody that specifically binds to Glial fibrillary acidic protein (“GFAP”) (or fragments thereof), referred to as “GFAP antibody.” The GFAP antibodies can be used to assess the GFAP status as a measure of traumatic brain injury, detect the presence of GFAP in a sample, quantify the amount of GFAP present in a sample, or detect the presence of and quantify the amount of GFAP in a sample.

a. Glial Fibrillary Acidic Protein (GFAP)

Glial fibrillary acidic protein (GFAP) is a 50 kDa intracytoplasmic filamentous protein that constitutes a portion of the cytoskeleton in astrocytes, and it has proved to be the most specific marker for cells of astrocytic origin. GFAP protein is encoded by the GFAP gene in humans. GFAP is the principal intermediate filament of mature astrocytes. In the central rod domain of the molecule, GFAP shares considerable structural homology with the other intermediate filaments. GFAP is involved in astrocyte motility and shape by providing structural stability to astrocytic processes. Glial fibrillary acidic protein and its breakdown products (GFAP-BDP) are brain-specific proteins released into the blood as part of the pathophysiological response after traumatic brain injury (TBI). Following injury to the human CNS caused by trauma, genetic disorders, or chemicals, astrocytes proliferate and show extensive hypertrophy of the cell body and processes, and GFAP is markedly upregulated. In contrast, with increasing astrocyte malignancy, there is a progressive loss of GFAP production. GFAP can also be detected in Schwann cells, enteric glia cells, salivary gland neoplasms, metastasizing renal carcinomas, epiglottic cartilage, pituicytes, immature oligodendrocytes, papillary meningiomas, and myoepithelial cells of the breast.

Human GFAP may have the following amino acid sequence:

(SEQ ID NO: 2)
MERRRITSAARRSYVSSGEMMVGGLAPGRRLGPGTRLSLARMPPPLPTR
VDFSLAGALNAGFKETRASERAEMMELNDRFASYIEKVRFLEQQNKALA
AELNQLRAKEPTKLADVYQAELRELRLRLDQLTANSARLEVERDNLAQD
LATVRQKLQDETNLRLEAENNLAAYRQEADEATLARLDLERKIESLEEE
IRFLRKIHEEEVRELQEQLARQQVHVELDVAKPDLTAALKEIRTQYEAM
ASSNMHEAEEWYRSKFADLTDAAARNAELLRQAKHEANDYRRQLQSLTC
DLESLRGTNESLERQMREQEERHVREAASYQEALARLEEEGQSLKDEMA
RHLQEYQDLLNVKLALDIEIATYRKLLEGEENRITIPVQTFSNLQIRET
SLDTKSVSEGHLKRNIVVKTVEMRDGEVIKESKQEHKDVM.

The human GFAP may be a fragment or variant of SEQ ID NO: 2. The fragment of GFAP may be between 5 and 400 amino acids, between 10 and 400 amino acids, between 50 and 400 amino acids, between 60 and 400 amino acids, between 65 and 400 amino acids, between 100 and 400 amino acids, between 150 and 400 amino acids, between 100 and 300 amino acids, or between 200 and 300 amino acids in length. The fragment may comprise a contiguous number of amino acids from SEQ ID NO: 2. The human GFAP fragment or variant of SEQ ID NO: 2 may be a GFAP breakdown product (BDP). The GFAP BDP may be 38 kDa, 42 kDa (fainter 41 kDa), 47 kDa (fainter 45 kDa); 25 kDa (fainter 23 kDa); 19 kDa, or 20 kDa. In some embodiments, the human GFAP fragment or variant can be a GFAP BDP comprising between 5 to 25 amino acids, between 5 to 50 amino acids, between 5 to 100 amino acids or 5 to 200 amino acids.

b. GFAP-Recognizing Antibody

The antibody is an antibody that binds to GFAP, a fragment thereof, an epitope of GFAP, or a variant thereof. The antibody may be a fragment of the anti-GFAP antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-GFAP antibodies may be a chimeric anti-GFAP or humanized anti-GFAP antibody. In one embodiment, both the humanized antibody and chimeric antibody are monovalent. In one embodiment, both the humanized antibody and chimeric antibody comprise a single Fab region linked to an Fe region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008(8):85. Therefore, the antibody may be a product of the human and not animal repertoire. Because it is of human origin, the risks of reactivity against self-antigens may be minimized. Alternatively, standard yeast display libraries and display technologies may be used to select and isolate human anti-GFAP antibodies. For example, libraries of naïve human single chain variable fragments (scFv) may be used to select human anti-GFAP antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to GFAP (SEQ ID NO: 2), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77: 4216-4220 (1980)), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human GFAP) and the other heavy and light chain are specific for an antigen other than human GFAP by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with GFAP or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes eletrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass); Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); Hawkins et al, J. Mol. Biol., 226: 889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al. (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (1311), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

### (1) Anti-GFAP Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with GFAP with myeloma cells and then screening the hybridomas resulting from the fusion for hybridoma clones that secrete an antibody able to bind a polypeptide of the disclosure. Briefly, rats can be immunized with a GFAP antigen. In a preferred embodiment, the GFAP antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a GFAP antigen, antibodies and/or antibody-producing cells may be obtained from the animal. An anti-GFAP antibody-containing serum is obtained from the animal by bleeding or sacrificing the animal. The serum may be used as it is obtained from the animal, an immunoglobulin fraction may be obtained from the serum, or the anti-GFAP antibodies may be purified from the serum. Serum or immunoglobulins obtained in this manner are polyclonal, thus having a heterogeneous array of properties.

Once an immune response is detected, e.g., antibodies specific for the antigen GFAP are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding GFAP. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed, and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using GFAP, or a portion thereof, or a cell expressing GFAP. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-GFAP antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-GFAP antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

### (2) Anti-GFAP Monoclonal Antibodies Using SLAM

In another embodiment of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93: 7843-7848 (1996). In this method, single cells secreting antibodies of interest, e.g., lymphocytes derived from any one of the immunized animals are screened using an antigen-specific hemolytic plaque assay, wherein the antigen GFAP, a subunit of GFAP, or a fragment thereof, is coupled to sheep red blood cells using a linker, such as biotin, and used to identify single cells that secrete antibodies with specificity for GFAP. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to GFAP. The amplified immunoglobulin sequences further can be manipulated in vitro, such as by in vitro affinity maturation method. See, for example, PCT Publication No. WO 97/29131 and PCT Publication No. WO 00/56772.

### (3) Anti-GFAP Monoclonal Antibodies Using Transgenic Animals

In another embodiment of the disclosure, antibodies are produced by immunizing a non-human animal comprising some, or all, of the human immunoglobulin locus with a GFAP antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7: 13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15: 146-156 (1997), Green and Jakobovits, J. Exp. Med., 188: 483-495 (1998), the disclosures of which are hereby incorporated by reference.

### (4) Anti-GFAP Monoclonal Antibodies Using Recombinant Antibody Libraries

In vitro methods also can be used to make the antibodies of the disclosure, wherein an antibody library is screened to identify an antibody having the desired GFAP-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9: 1369-1372 (1991); Hay et al., Hum. Antibod. Hybridomas, 3: 81-85 (1992); Huse et al., Science, 246: 1275-1281 (1989); McCafferty et al., Nature, 348: 552-554 (1990); Griffiths et al., EMBO J., 12: 725-734 (1993); Hawkins et al., J. Mol. Biol., 226: 889-896 (1992); Clackson et al., Nature, 352: 624-628 (1991); Gram et al., Proc. Natl. Acad. Sci. USA, 89: 3576-3580 (1992); Garrard et al., Bio/Technology, 9: 1373-1377 (1991); Hoogenboom et al., Nucl. Acids Res., 19: 4133-4137 (1991); Barbas et al., Proc. Natl. Acad. Sci. USA, 88: 7978-7982 (1991); U.S. Patent Application Publication No. 2003/0186374; and PCT Publication No. WO 97/29131, the contents of each of which are incorporated herein by reference.

The recombinant antibody library may be from a subject immunized with GFAP, or a portion of GFAP. Alternatively, the recombinant antibody library may be from a naive subject, i.e., one who has not been immunized with GFAP, such as a human antibody library from a human subject who has not been immunized with human GFAP. Antibodies of the disclosure are selected by screening the recombinant antibody library with the peptide comprising human GFAP to thereby select those antibodies that recognize GFAP. Methods for conducting such screening and selection are well known in the art, such as described in the references in the preceding paragraph. To select antibodies of the disclosure having particular binding affinities for GFAP, such as those that dissociate from human GFAP with a particular K_{off} rate constant, the art-known method of surface plasmon resonance can be used to select antibodies having the desired K_{off} rate constant. To select antibodies of the disclosure having a particular neutralizing activity for GFAP, such as those with a particular IC₅₀, standard methods known in the art for assessing the inhibition of GFAP activity may be used.

In one embodiment, the disclosure pertains to an isolated antibody, or an antigen-binding portion thereof, that binds human GFAP. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182: 41-50 (1995); Ames et al., J. Immunol. Methods, 184:177-186 (1995); Kettleborough et al., Eur. J. Immunol., 24: 952-958 (1994); Persic et al., Gene, 187: 9-18 (1997); Burton et al., Advances in Immunology, 57: 191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12(6): 864-869 (1992); Sawai et al., Am. J. Reprod. Immunol., 34: 26-34 (1995); and Better et al., Science, 240: 1041-1043 (1988). Examples of techniques which can be used to produce single-chain Fvs and antibodies include those described in U.S. Pat. Nos. 4,946,778 and 5,258,498; Huston et al., Methods in Enzymology, 203: 46-88 (1991); Shu et al., Proc. Natl. Acad. Sci. USA, 90: 7995-7999 (1993); and Skerra et al., Science, 240: 1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94: 12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant GFAP Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77: 4216-4220 (1980), used with a DHFR selectable marker, e.g., as described in Kaufman and Sharp, J. Mol. Biol., 159: 601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human GFAP) and the other heavy and light chain are specific for an antigen other than human GFAP by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for GFAP and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for GFAP, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g. mice that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-GFAP Antibodies

Anti-GFAP antibodies may be generated using the techniques described above as well as using routine techniques known in the art. In some embodiments, the anti-GFAP antibody may be an unconjugated GFAP antibody, such as GFAP antibodies available from Dako (Catalog Number: M0761), ThermoFisher Scientific (Catalog Numbers: MA5-2023, A-21282, 13-0300, MA1-19170, MA1-19395, MA5-15086, MA5-16367, MA1-35377, MA1-06701, or MA1-20035), AbCam (Catalog Numbers: ab10062, ab4648, ab68428, ab33922, ab207165, ab190288, ab115898, or ab21837), EMD Millipore (Catalog Numbers: FCMAB257P, MAB360, MAB3402, 04-1031, 04-1062, MAB5628), Santa Cruz (Catalog Numbers: sc-166481, sc-166458, sc-58766, sc-56395, sc-51908, sc-135921, sc-71143, sc-65343, or sc-33673), Sigma-Aldrich (Catalog Numbers: G3893 or G6171) or Sino Biological Inc. (Catalog Number: 100140-R012-50). The anti-GFAP antibody may be conjugated to a fluorophore, such as conjugated GFAP antibodies available from ThermoFisher Scientific (Catalog Numbers: A-21295 or A-21294), EMD Millipore (Catalog Numbers: MAB3402X, MAB3402B, MAB3402B, or MAB3402C3) or AbCam (Catalog Numbers: ab49874 or ab194325).

Alternatively, the antibodies described in WO 2018/067474, WO2018/081649, U.S. Pat. No. 11,078,298, U.S. Patent Publication No. 2019/0502127, and/or Bazarian et al., “Accuracy of a rapid GFAP/UCH-L1 test for the prediction of intracranial injuries on head CT after mild traumatic brain injury”, Acad. Emerg. Med., (Aug. 6, 2021), the contents of which are herein incorporated by reference, can also be used.

### 7. Other Factors

The methods of diagnosing, prognosticating, and/or assessing, as described above, can further include using other factors for the diagnosis, prognostication, and assessment. In some embodiments, traumatic brain injury may be diagnosed using the Glasgow Coma Scale or the Extended Glasgow Outcome Scale (GOSE). Other tests, scales or indices can also be used either alone or in combination with the Glasgow Coma Scale. An example is the Ranchos Los Amigos Scale. The Ranchos Los Amigos Scale measures the levels of awareness, cognition, behavior and interaction with the environment. The Ranchos Los Amigos Scale includes: Level I: No Response; Level II: Generalized Response; Level III: Localized Response; Level IV: Confused-agitated; Level V: Confused-inappropriate; Level VI: Confused-appropriate; Level VII: Automatic-appropriate; and Level VIII: Purposeful-appropriate. Another example is the Rivermead Post-Concussion Symptoms Questionairre, a self-report scale to measure the severity of post-concussive symptoms following TBI. Patients are asked to rate how severe each of 16 symptoms (e.g., headache, dizziness, nausea, vomiting) has been over the past 24 hours. In each case, the symptom is compared with how severe it was before the injury occurred (premorbid). These symptoms are reported by severity on a scale from 0 to 4: not experienced, no more of a problem, mild problem, moderate problem, and severe problem.

### 8. Samples

In some embodiments, the sample is obtained from a subject (e.g., human subject) that has sustained an injury or is suspected of having sustained an injury to the head that may have been or has been caused by any one or combination of factors. In some embodiments, the sample is obtained after the subject sustained an injury to the head caused by physical shaking, blunt impact by an external mechanical or other force that results in a closed or open head trauma, one or more falls, explosions or blasts or other types of blunt force trauma. In some embodiments, the sample is obtained after the subject has ingested or been exposed to a fire, chemical, or toxin, or a combination of a fire, chemical and/or toxin. Examples of such chemicals and/or toxins include molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. In some embodiments, the sample is obtained from a subject that suffers from an autoimmune disease, a metabolic disorder, a brain tumor, hypoxia, a viral infection (SARS-CoV-2), a fungal infection, a bacterial infection, meningitis, hydrocephalus, or any combinations thereof.

In yet another embodiment, the methods described herein use samples that also can be used to determine whether or not a subject has or is at risk of developing a TBI (such as a mild TBI, moderate TBI, severe TBI, or moderate to severe TBI) by determining the levels of UCH-L1 and/or GFAP in a subject using the anti-UCH-L1 and/or anti-GFAP antibodies described below, or antibody fragments thereof. Thus, in particular embodiments, the disclosure also provides a method for determining whether a subject having, or at risk for, traumatic brain injuries, discussed herein and known in the art, is a candidate for therapy or treatment. Generally, the subject is at least one who: (i) has experienced an injury to the head; (ii) ingested and/or been exposed to one or more chemicals and/or toxins; (iii) suffers from an autoimmune disease, a metabolic disorder, a brain tumor, hypoxia, a viral infection (e.g., SARS-CoV-2), a fungal infection, a bacterial infection, meningitis, hydrocephalus, or any combinations thereof; or (iv) any combinations of (i)-(iii); or, who has actually been diagnosed as having, or being at risk for TBI (such as, for example, subjects suffering from an autoimmune disease, a metabolic disorder, a brain tumor, hypoxia, a viral infection (e.g., SARS-CoV-2), a fungal infection, a bacterial infection, meningitis, hydrocephalus, or any combinations thereof), and/or who demonstrates an unfavorable (i.e., clinically undesirable) concentration or amount of UCH-L1 and/or GFAP or UCH-L1 and/or GFAP fragment, as described herein.

### A. Test or Biological Sample

As used herein, “sample”, “test sample”, “biological sample” refer to fluid sample containing or suspected of containing GFAP and/or UCH-L1. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing GFAP and/or UCH-L1 may be assayed directly. In a particular example, the source containing GFAP and/or UCH-L1 is a human (e.g., pediatric or adult human) substance or substance from another species. As used herein, the term “pediatric” or “pediatric subject” refers to a subject less than 18 years of age (i.e., not 18 years of age or older). For example, a pediatric subject may be less than about 18 years old, or about 17 years old, about 16 years old, about 15 years old, about 14 years old, about 13 years old, about 12 years old, about 11 years old, about 10 years old, about 9 years old, about 8 years old, about 7 years old, about 6 years old, about 5 years old, about 4 years old, about 3 years old, about 2 years old, about 1 year old, or less than about 1 year old. In some embodiments, the pediatric subject may be less than about 1 year old to about less than 18 years old. In some embodiment, the pediatric subject may be less than about 1 year old to about 17 years old. For example, a pediatric subject may be anywhere from about one day, about two days, about three days, about four days, about five days, about six days, about one week, about two weeks, about three weeks, about one month, about two months, about three months, about four months, about five months, about six months, about seven months, about eight months, about nine months, about ten months, or about eleven months, in total, less than: about 18 years old, or about 17 years old, or about 16 years old, or about 15 years old, or about 14 years old, or about 13 years old, or about 12 years old, or about 11 years old, or about 10 years old, or about 9 years old, or about 8 years old, or about 7 years old, or about 6 years old, or about 5 years old, or about 4 years old, or about 3 years old, or about 2 years old, or about 1 year old, or less than about 1 year old. An “adult” or an “adult subject” refers to a subject 18 years of age or older.

The substance optionally is a bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. In some embodiments, the sample is a whole blood sample, a serum sample, a cerebrospinal fluid sample, a mixed sample of venous and capillary blood, a mixed sample of capillary blood and interstitial fluid, a tissue sample, a bodily fluid, or a plasma sample.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source containing GFAP and/or UCH-L1 is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source containing GFAP and/or UCH-L1 is a body fluid (e.g., blood, serum) from a subject (e.g., human or other species), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

### 9. Kits

Provided herein is a kit, which may be used for assaying or assessing a test sample for UCH-L1 and/or GFAP or UCH-L1 and/or GFAP fragment. The kit comprises at least one lateral flow device for assaying the test sample for UCH-L1 and/or GFAP. In some embodiments, the kit comprises a plurality (e.g., two) of lateral flow devices individually wrapped in moisture impervious wrapping and packaged together. In addition to at least one lateral flow device, the kit can also contain instructions for assaying the test sample for UCH-L1 and/or GFAP. For example, the kit can comprise instructions for assaying the test sample for UCH-L1 and/or GFAP using the lateral flow devices described herein. Instructions included in kits can be affixed to packaging material or can be included as a package insert. While the instructions are typically written or printed materials, they are not limited to such. For example, in some embodiments, a bar code (e.g., QR code) can be provided on the lateral flow device, packing material or package insert which can be scanned by a mobile device (e.g., phone, iPad, watch) to view the instructions. In fact, any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Alternatively or additionally, the kit can comprise a calibrator or control, e.g., purified, and optionally lyophilized, UCH-L1 and/or GFAP, and/or at least one container (e.g., tube, microtiter plates or strips, which can be already coated with an anti-UCH-L1 and/or GFAP monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., lateral flow devices, reagents, standards, buffers, diluents, etc., which are necessary to perform the assay.

The kit can also optionally include other reagents required to conduct an assay, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary. The kit can further include containers for holding or storing a sample (e.g., a container or cartridge for a urine, whole blood, plasma, or serum sample). Where appropriate, the kit optionally also can contain mixing vessels, and other components that facilitate the preparation of reagents or the test sample. The kit can also include one or more instruments for assisting with obtaining a test sample, such as a syringe, pipette, forceps, measured spoon, or the like.

If desired, the kit can further comprise one or more components, alone or in further combination with instructions, for assaying the test sample for another analyte, which can be a biomarker.

### 10. Examples

It will be readily apparent to those skilled in the art that other suitable modifications and adaptations of the methods of the present disclosure described herein are readily applicable and appreciable, and may be made using suitable equivalents without departing from the scope of the present disclosure or the embodiments disclosed herein. Having now described the present disclosure in detail, the same will be more clearly understood by reference to the following examples, which are merely intended only to illustrate some embodiments of the disclosure, and should not be viewed as limiting to the scope of the disclosure. The disclosures of all journal references, U.S. patents, and publications referred to herein are hereby incorporated by reference in their entireties.

The present disclosure has multiple embodiments, illustrated by the following non-limiting examples.

### Example 1

### GFAP Lateral Flow Assay

An exemplary lateral flow assay for detecting GFAP is provided that uses a sandwich-type assay. In particular, the lateral flow device comprises a first zone (e.g., a reagent zone) comprising a Fab monoclonal antibody that is specific for a first epitope on GFAP and is labeled with a detectable label, such as, a colloidal metal, such as colloidal gold. The device comprises an immobilized monoclonal antibody that is specific for a second, different epitope of GFAP at a second zone (e.g., a detection zone). A positive test indicating the presence of GFAP indicated by the appearance of a visible line at the detection zone (e.g., at a test line).

Monoclonal antibody pairs, such as Antibody A as a capture monoclonal antibody and Antibody B as a detection monoclonal antibody, were used. Antibody A and Antibody B are exemplary anti-GFAP antibodies that were internally developed at Abbott Laboratories (Abbott Park, IL). Antibody A and Antibody B bind to epitopes within the same GFAP breakdown product. Other commercially available antibodies, such as those described previously herein, can be used together as capture antibodies or detection antibodies, in various combinations in such a lateral flow device. Optionally, any form, combination and/or number of antibodies can be used. For example, all antibodies used can be monoclonal antibodies, all antibodies employed can be Fabs, alternatively, there can be a mixture of monoclonal antibodies and Fabs. Two antibodies can be used, three antibodies could be used, four antibodies could be used, etc.

Recombinant GFAP was spiked (about 40 μg/mL to about 1000 μg/mL) into a buffer solution to provide a sample volume of 80 μL. A portion of the sample was applied to the first zone of the exemplary lateral flow device. A positive test indicating the presence of GFAP was indicated within 15 minutes by the appearance of a visible line at the detection zone. The visual limit of detection was determined to be approximately 125 μg/mL. This visual limit of detection corresponds to an internally assigned Immunochromatography (ICT) score of 0.5.

The ICT score was employed solely to assist with standardizing results across experiments and understanding the sensitivity limit of the test that may enable a darker line and a higher incidence of positive reads. Essentially for assigning scores an ICT scorecard was developed corresponding to a gradient of value, with the lightness or darkness of a visible line that might be observed at the detection zone shown in stepped values of pink or another color, and a number assigned increasing with increasing darkness of the value or saturation/intensity of the color. Generally for an ICT score=0.5, it is understood that naïve operators (i.e., those that are not experienced at reviewing visible lines) can read between 70-80% of lines developed. The same naïve operators who could not see an ICT score of 0.5 typically could see higher intensity lines at ICT scores of 0.5 or 1. The naïve operators could see 10-20% of lines developed at ICT score=0.25. In contrast, 100% of ‘experienced’ operators (i.e., those experienced at reviewing visible lines, such as internal R&D and QC operators) could see lines developed at ICT=0.5 or lower.

Additionally, the signal provided by recombinant GFAP spiked into the buffer between about 40 μg/mL to about 1000 μg/mL was found to be linear across the dilutions tested at ICT scores between 0.25 to 2.5 (including a zero control).

### Example 2

### UCH-L1 Lateral Flow Assay

An exemplary lateral flow assay for detecting UCH-L1 is provided that uses a sandwich-type assay. In particular, the lateral flow device comprises a first zone (e.g., a reagent zone) comprising a monoclonal antibody that is specific for a first epitope on UCH-L1 and is labeled with a detectable label. The device comprises an immobilized monoclonal antibody that is specific for a second, different epitope of UCH-L1 at a second zone (e.g., a detection zone). A positive test indicating the presence of UCH-L1 indicated by the appearance of a visible line at the detection zone (e.g., at a test line).

Monoclonal antibody pairs, such as Antibody A as a capture monoclonal antibody and Antibody B as a detection monoclonal antibody, were used. Antibody A is an exemplary anti-UCH-L1 antibody that was internally developed at Abbott Laboratories (Abbott Park, IL). Antibody B recognizes a different epitope of UCH-L1 and was developed by Banyan Biomarkers (Alachua, Florida). Other antibodies that were internally developed at Abbott Laboratories (Abbott Park, IL), or other commercially available antibodies, such as those described herein, can be used together as capture antibodies or detection antibodies, in various combinations in such a lateral flow device. Optionally, any form, combination and/or number of antibodies can be used. For example, all antibodies used can be monoclonal antibodies, all antibodies employed can be Fabs, alternatively, there can be a mixture of monoclonal antibodies and Fabs. Two antibodies can be used, three antibodies could be used, four antibodies could be used, etc.

Recombinant UCH-L1 was spiked (about 400 μg/mL to about 20,000 μg/mL) into a buffer solution to provide a sample volume of 80 μL. A portion of the sample was applied to the first zone of the exemplary lateral flow device. A positive test indicating the presence of UCH-L1 was indicated within 15 minutes by the appearance of a visible line at the detection zone. The visual limit of detection (ICT score=0.5) was determined to be approximately 1250 μg/mL. Additionally, the signal provided by recombinant UCH-L1 spiked into the buffer between about 400 μg/mL to about 20,000 μg/mL was found to be linear at ICT scores between 0.25 to 2.5 (including a zero control).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various embodiments of the disclosure are set out in the following numbered clauses:

For reasons of completeness, various embodiments of the disclosure are set out in the following numbered clauses:

Clause 1. A method comprising:
performing at least one lateral flow assay on a biological sample obtained from a subject to determine an amount or presence of ubiquitin carboxy-terminal hydrolase L1 (UCH-L1) alone, or an amount or presence of UCH-L1 and an amount or presence of glial fibrillary acidic protein (GFAP); and
displaying the amount or presence of UCH-L1 alone or UCH-L1 and GFAP determined in the sample.

Clause 2. The method of clause 1, wherein the at least one lateral flow assay is part of a lateral flow device.

Clause 3. The method of clause 2, wherein the lateral flow device comprises (a) at least one test strip; or (b) at least two test strips.

Clause 4. The method of any of clauses 1-3, wherein (a) the lateral flow assay for UCH-L1 comprises a first specific binding partner which binds to an epitope on UCH-L1 and a second specific binding partner comprising a detectable label; and (b) the lateral flow assay for GFAP comprises a third specific binding partner which binds to an epitope on GFAP and a fourth specific binding partner comprising a detectable label.

Clause 5. The method of clause 4, wherein the first specific binding partner, second specific binding partner, third specific binding partner, fourth specific binding partner, or any combination thereof is an antibody or antibody fragment thereof.

Clause 6. The method of any of clauses 1-5, wherein the method is used to aid in a diagnosis and evaluation of a subject that has sustained or may have sustained an injury to the head.

Clause 7. The method of clause 6, wherein the subject is diagnosed as having a traumatic brain injury.

Clause 8. The method of clause 7, subject is diagnosed as having a mild, moderate, severe, or moderate to severe traumatic brain injury.

Clause 9. The method of any of clauses 1-8, wherein the biological sample is the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 10. The method of any of clauses 1-9, wherein the method is used to determine whether a subject should receive a head computed tomography (CT) scan, a magnetic resonance imaging (MRI) scan or both a head CT scan and a MRI scan.

Clause 11. A method comprising:
performing at least one lateral flow assay on a biological sample obtained from a subject to determine an amount or presence of ubiquitin carboxy-terminal hydrolase L1 (UCH-L1), glial fibrillary acidic protein (GFAP) or both UCH-L1 and GFAP; and
visualizing the amount or presence of UCH-L1, GFAP, UCH-L1 and GFAP determined in the sample,
wherein the assay does not require a device to read the amount or presence of UCH-L1, GFAP, or UCH-L1 and GFAP.

Clause 12. The method of clause 11, wherein (a) the lateral flow assay for UCH-L1 comprises a first specific binding partner which binds to an epitope on UCH-L1 and a second specific binding partner comprising a detectable label; and (b) the lateral flow assay for GFAP comprises a third specific binding partner which binds to an epitope on GFAP and a fourth specific binding partner comprising a detectable label.

Clause 13. The method of clause 12, wherein the first specific binding partner, second specific binding partner, third specific binding partner, fourth specific binding partner, or any combination thereof is an antibody or antibody fragment thereof.

Clause 14. The method of any of clauses 11-13, wherein the method is used to aid in a diagnosis and evaluation of a subject that has sustained or may have sustained an injury to the head.

Clause 15. The method of clause 14, wherein the subject is diagnosed as having a traumatic brain injury.

Clause 16. The method of clause 15, subject is diagnosed as having a mild, moderate, severe, or moderate to severe traumatic brain injury.

Clause 17. The method of any of clauses 11-16, wherein the biological sample is the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

Clause 18. The method of any of clauses 11-17, wherein the method is used to determine whether a subject should receive a head computed tomography (CT) scan, a magnetic resonance imaging (MRI) scan or both a head CT scan and a MRI scan.

Clause 19. The method of any of clauses 12-18, wherein the detectable label is a colloidal metal particle, a colloidal non-metal particle, a latex particle, a color or dyed particle, or any combinations thereof.

Clause 20. The method of clause 19, wherein the detectable label is a colloidal metal particle.

Clause 21. The method of clause 19, wherein the colloidal metal particle is a gold or silver colloidal particle.

Clause 22. The method of any of clauses 12-21, wherein the detectable label is visible by a naked eye.

Clause 23. A kit for performing the method of clauses 1 or 11, wherein the kit comprises,
a first lateral flow device for detecting a presence of ubiquitin carboxy-terminal hydrolase L1 (UCH-L1) in the sample;
a second lateral flow device for detecting a presence of glial fibrillary acidic protein (GFAP) in the sample; and
instructions for detecting the presence of UCH-L1 and GFAP in the sample.

Clause 24. A kit for performing the method of clauses 1 or 11, wherein the kit comprises,
a lateral flow device for detecting a presence of ubiquitin carboxy-terminal hydrolase L1 (UCH-L1) and glial fibrillary acidic protein (GFAP) in the sample; and
instructions for detecting the presence of UCH-L1 and GFAP in the sample.

Clause 25. The kit according to clause 24, wherein the kit comprises at least one test strip.

Clause 26. The kit according to clause 24, wherein the kit comprises at least two test strips.

Clause 27. The method of any of clauses 1-22, wherein the at least one lateral flow assay for GFAP, the at least one lateral flow assay for UCH-L1, or at least one lateral flow assay for GFAP and at least one lateral flow assay for UCH-L1 are each performed or capable of being performed in less than about 30 minutes, less than about 25 minutes, less than about 20 minutes, less than about 18 minutes, or less than about 15 minutes.

Clause 28. The method of any of clauses 1-22, wherein the at least one lateral flow assay for GFAP, the at least one lateral flow assay for UCH-L1, or at least one lateral flow assay for GFAP and at least one lateral flow assay for UCH-L1 are each are each performed or capable of being performed in a time ranging from about 4 to about 20 minutes, from about 10 to about 15 minutes or from about 15 to about 18 minutes.

What is claimed is:

1. A method comprising:
performing at least one lateral flow assay on a biological sample obtained from a subject to determine an amount or presence of ubiquitin carboxy-terminal hydrolase L1 (UCH-L1) alone, or an amount or presence of UCH-L1 and an amount or presence of glial fibrillary acidic protein (GFAP); and
displaying the amount or presence of UCH-L1 alone or UCH-L1 and GFAP determined in the sample.

2. The method of claim 1, wherein the at least one lateral flow assay is part of a lateral flow device.

3. The method of claim 2, wherein the lateral flow device comprises (a) at least one test strip; or (b) at least two test strips.

4. The method of claim 1, wherein (a) the lateral flow assay for UCH-L1 comprises a first specific binding partner which binds to an epitope on UCH-L1 and a second specific binding partner comprising a detectable label; and (b) the lateral flow assay for GFAP comprises a third specific binding partner which binds to an epitope on GFAP and a fourth specific binding partner comprising a detectable label.

5. The method of claim 4, wherein the first specific binding partner, second specific binding partner, third specific binding partner, fourth specific binding partner, or any combination thereof is an antibody or antibody fragment thereof.

6. The method of claim 1, wherein the method is used to aid in a diagnosis and evaluation of a subject that has sustained or may have sustained an injury to the head.

7. The method of claim 6, wherein the subject is diagnosed as having a traumatic brain injury.

8. The method of claim 7, subject is diagnosed as having a mild, moderate, severe, or moderate to severe traumatic brain injury.

9. The method of claim 1, wherein the biological sample is the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

10. The method of claim 1, wherein the method is used to determine whether a subject should receive a head computed tomography (CT) scan, a magnetic resonance imaging (MRI) scan or both a head CT scan and a MRI scan.

11. A method comprising:
performing at least one lateral flow assay on a biological sample obtained from a subject to determine an amount or presence of ubiquitin carboxy-terminal hydrolase L1 (UCH-L1), glial fibrillary acidic protein (GFAP) or both UCH-L1 and GFAP; and
visualizing the amount or presence of UCH-L1, GFAP, UCH-L1 and GFAP determined in the sample,
wherein the assay does not require a device to read the amount or presence of UCH-L1, GFAP, or UCH-L1 and GFAP

12. The method of claim 11, wherein (a) the lateral flow assay for UCH-L1 comprises a first specific binding partner which binds to an epitope on UCH-L1 and a second specific binding partner comprising a detectable label; and (b) the lateral flow assay for GFAP comprises a third specific binding partner which binds to an epitope on GFAP and a fourth specific binding partner comprising a detectable label.

13. The method of claim 12, wherein the first specific binding partner, second specific binding partner, third specific binding partner, fourth specific binding partner, or any combination thereof is an antibody or antibody fragment thereof.

14. The method of claim 11, wherein the method is used to aid in a diagnosis and evaluation of a subject that has sustained or may have sustained an injury to the head.

15. The method of claim 14, wherein the subject is diagnosed as having a traumatic brain injury.

16. The method of claim 15, subject is diagnosed as having a mild, moderate, severe, or moderate to severe traumatic brain injury.

17. The method of claim 11, wherein the biological sample is the sample is selected from the group consisting of a blood sample, a urine sample, a cerebrospinal fluid sample, a tissue sample, a bodily fluid sample, a saliva sample, an oropharyngeal specimen, and a nasopharyngeal specimen.

18. The method of claim 11, wherein the method is used to determine whether a subject should receive a head computed tomography (CT) scan, a magnetic resonance imaging (MRI) scan or both a head CT scan and a MRI scan.

19. The method of claim 12, wherein the detectable label is a colloidal metal particle, a colloidal non-metal particle, a latex particle, a color or dyed particle, or any combinations thereof.

20. The method of claim 19, wherein the detectable label is a colloidal metal particle.

21. The method of claim 20, wherein the colloidal metal particle is a gold or silver colloidal particle.

22. The method of claim 12, wherein the detectable label is visible by a naked eye.

23. A kit for performing the method of claim 1, wherein the kit comprises,
a first lateral flow device for detecting a presence of ubiquitin carboxy-terminal hydrolase L1 (UCH-L1) in the sample;
a second lateral flow device for detecting a presence of glial fibrillary acidic protein (GFAP) in the sample; and
instructions for detecting the presence of UCH-L1 and GFAP in the sample.

24. A kit for performing the method of claim 1, wherein the kit comprises,
a lateral flow device for detecting a presence of ubiquitin carboxy-terminal hydrolase L1 (UCH-L1) and glial fibrillary acidic protein (GFAP) in the sample; and
instructions for detecting the presence of UCH-L1 and GFAP in the sample.

25. The kit according to claim 24, wherein the kit comprises at least one test strip.

26. The kit according to claim 24, wherein the kit comprises at least two test strips.

27. The method of claim 1, wherein the at least one lateral flow assay for GFAP, the at least one lateral flow assay for UCH-L1, or at least one lateral flow assay for GFAP and at least one lateral flow assay for UCH-L1 are each performed or capable of being performed in less than about 30 minutes, less than about 25 minutes, less than about 20 minutes, less than about 18 minutes, or less than about 15 minutes.

28. The method of claim 1, wherein the at least one lateral flow assay for GFAP, the at least one lateral flow assay for UCH-L1, or at least one lateral flow assay for GFAP and at least one lateral flow assay for UCH-L1 are each are each performed or capable of being performed in a time ranging from about 4 to about 20 minutes, from about 10 to about 15 minutes or from about 15 to about 18 minutes.
